(19) United States
(12) Patent Application Publication
(10) Pub. No.: US 2026/0273141 A1
(43) Pub. Date: Sep. 17, 2026

(54) Methods For Inhibiting Neuronal Degeneration To Preserve Residual Functionality And Enhance Axonal Fusion

(71) Applicants: United States Government As Represented By The Department Of Veterans Affairs, Washington, DC (US); The Trustees of the University of Pennsylvania, Philadelphia, PA (US)

(72) Inventors: Justin C. Burrell, Philadelphia, PA (US); D. Kacy Cullen, Philadelphia, PA (US); Christopher Cardozo, Bronx, NY (US); Carlos Toro Chacon, Bronx, NY (US); Tomisin Latona, Philadelphia, PA (US)

(21) Appl. No.: 19/567,985

(22) Filed: Mar. 16, 2026

Related U.S. Application Data
(60) Provisional application No. 63/772,164, filed on Mar. 14, 2025.

(51) Int. Cl. A61L 27/54; A01N 1/126; A61L 27/22; A61L 27/24; A61L 27/36; A61L 27/52; C08L 89/06; C12N 5/00; C12N 5/071; C12N 9/99; G01N 33/68

(52) U.S. Cl. CPC A61L 27/54; A01N 1/126; A61L 27/225; A61L 27/24; A61L 27/3633; A61L 27/52; C08L 89/06; C12N 5/0018; C12N 5/0697; C12N 9/99; G01N 33/6896; A61L 2300/204; A61L 2400/06; A61L 2430/32; C12N 2501/73; G01N 2333/46

(57) ABSTRACT

Disclosed herein are methods of inhibiting sterile alpha and TIR motif-containing protein 1 (SARM1) NADase activity in an axon. The methods can also be used to reduce or delay degeneration in an axon, treat or prevent degeneration of an injured axon, enhance fusion of one or more severed axons, and delay degeneration of one or more fused axons, as well as treat a neurological nerve injury or a neurodegenerative disease in a subject in need thereof using a SARM 1 inhibitor.

### CROSS-REFERENCE TO RELATED APPLICATIONS

This application claims benefit of the filing date of U.S. Provisional Application No. 63/772,164, filed Mar. 14, 2025. The content of this application is hereby incorporated herein by reference in its entirety.

### STATEMENT REGARDING FEDERALLY SPONSORED RESEARCH

This invention was made with government support under Grant Numbers TL1-TR001880, T90-DE030854, R44-NS125892, R01-NS117757 awarded by the National Institutes of Health, OR230229 awarded by the Department of Defense, and I01-RX005045 awarded by the Department of Veterans Affairs. The government has certain rights in this invention.

### BACKGROUND

Slow axon regeneration is often considered the primary limitation to successful functional recovery after peripheral nerve injury (PNI). However, changes in proximal neurons, distal nerve segments, and distal end targets also substantially contribute to the likelihood, timing, and extent of functional recovery.

Traumatic injury to the nervous system often results in debilitating sensorimotor deficits that negatively affect the patient's quality of life, such as limb paralysis, sensory loss, and/or intractable neuropathic pain. PNI has been reported to have a prevalence of almost 3% in level 1 trauma patients. In total, the annual number of PNI procedures in the U.S. is estimated to be 558,862. Despite recent advancements in peripheral nerve surgery, approximately 50% of patients recover functionality from the sensorimotor deficits. Thus, new treatments are needed.

### SUMMARY

Disclosed herein are methods of inhibiting sterile alpha and TIR motif-containing protein 1 (SARM1) NADase activity in an axon, the methods comprising: contacting the axon with an effective amount of a SARM1 inhibitor, thereby inhibiting SARM1 NADase activity in the axon.

Disclosed herein are methods of reducing or delaying degeneration in an axon, the methods comprising: contacting the axon with an effective amount of a sterile alpha and TIR motif-containing protein 1 (SARM1) inhibitor, thereby reducing or delaying degeneration of the axon

Disclosed herein are methods of treating or preventing degeneration of an injured axon, the methods comprising: contacting a neural tissue comprising the axon ex vivo with an effective amount of a sterile alpha and TIR motif-containing protein 1 (SARM1) inhibitor, thereby treating or preventing degeneration in the injured axon.

Disclosed herein are methods of enhancing fusion of one or more severed axons, the methods comprising: (a) contacting ex vivo a neural tissue comprising the one or more severed axons with an effective amount of a sterile alpha and TIR motif-containing protein 1 (SARM1) inhibitor; and (b) contacting ex vivo the neural tissue comprising the one or more severed axons in (a) with an effective amount of a fusogen, wherein the contacting in (a) is prior to or concurrent with the contacting of (b), thereby enhancing the fusion of the one or more severed axons.

Disclosed herein are methods of delaying degeneration of one or more fused axons, the methods comprising: (a) contacting ex vivo a neural tissue comprising the two or more fused axons with an effective amount of a sterile alpha and TIR motif-containing protein 1 (SARM1) inhibitor; and (b) contacting ex vivo the neural tissue comprising the one or more fused axons in (a) with an effective amount of a fusogen, wherein the contacting in (a) is prior to or concurrent with the contacting of (b), thereby delaying degeneration of the one or more severed axons.

Disclosed herein are methods of enhancing fusion of one or more severed axons and/or delaying degeneration of the subsequently fused axons, the method comprising: (a) contacting ex vivo a neural tissue comprising the two or more severed axons with an effective amount of a sterile alpha and TIR motif-containing protein 1 (SARM1) inhibitor; and (b) contacting ex vivo the neural tissue comprising the one or more severed axons in (a) with an effective amount of a fusogen, wherein the contacting in (a) is prior to or concurrent with the contacting of (b), thereby enhancing the fusion of the one or more severed axons and/or delaying degeneration of the subsequently fused axons.

Disclosed herein are methods of treating a neurological nerve injury or a neurodegenerative disease in a subject in need thereof, the methods comprising: administering to the subject an effective amount of a sterile alpha and TIR motif-containing protein 1 (SARM1) inhibitor, thereby treating a neurological injury or a neurodegenerative disease in the subject.

Disclosed herein are method of preserving axonal integrity within an engineered neural tissue construct, the methods comprising contacting the engineered neural tissue construct with an effective amount of a sterile alpha and TIR motif-containing protein 1 (SARM1) inhibitor, thereby maintaining axonal continuity within the construct prior to implantation into a subject.

Disclosed herein are compositions comprising a sterile alpha and TIR motif-containing protein 1 (SARM1) inhibitor and a biomaterial matrix.

Other features and advantages of the present compositions and methods are illustrated in the description below, the drawings, and the claims.

### BRIEF DESCRIPTION OF THE DRAWINGS

FIGS. 1A-C show schematics of uninjured and injured nerves. FIG. 1A shows that in healthy, uninjured nerve, axons extend from the proximal neurons either near or within the spinal cord and innervate the muscle and/or sensory end targets. Muscle targets are shown as an example in this schematic. Schwann cells are glial support cells within the nerve and are useful for axon maturation and myelination. A consideration for functional recovery prognosis is the nerve injury severity and the extent of damage to the underlying nerve architecture. FIG. 1B shows crush or compression nerve injury is the mildest form of nerve injury where the underlying nerve architecture remains intact that either spares the axons (neurapraxia) or results in axon degradation (axonotmesis). Immediately after injury, muscle function is diminished (as shown in yellow) and may return without surgical intervention. FIG. 1C shows that after nerve transection, Wallerian degeneration and surgical reconstruction are useful for functional recovery. The most challenging clinical scenarios with the worst prognoses are cases with segmental nerve injury, with gap lengths greater than 3 cm. Nerve reconstruction (in orange) can be achieved with various scaffolds, such as autograft, nerve guidance tube, or acellular nerve allograft. Distal nerve transfer is also an option.

FIG. 2 shows examples of various nerve injury locations. Another consideration for functional recovery is location of nerve injury. In an uninjured nerve, Schwann cells surround the axons and provide support and myelination. Severe injury resulting in Wallerian degeneration leads to Schwann cell detachment, migration, and an elevated pro-regenerative phenotype. Distance plays an role in functional recovery because longer regenerative distances to the end target result in prolonged periods of denervation, which negatively impacts the regenerative capacity of the distal Schwann cells and muscles. Additionally, location of the injury impacts proximal neuron survival. For example, the risk of motor neuron loss increases after ventral avulsion and/or increased proximal injury compared to distal injury.

FIGS. 3A-C show schematics of early nerve repair. FIG. 3A show that shortly after segmental injury, axons in the distal nerve undergo Wallerian degeneration and breakdown, and the Schwann cells align into a temporary pro-regenerative phenotype. FIG. 3B shows that for nerve repairs completed in the acute injury phase, axons use the pro-regenerative Schwann cells to extend through the otherwise denervated distal nerve. FIG. 3C shows that early nerve repair increases the likelihood of greater muscle reinnervation, with increased prognosis for functional recovery.

FIGS. 4A-C shows schematics of delayed nerve repair. FIG. 4A shows that after segmental injury, axons in the distal nerve undergo Wallerian degeneration. Schwann cells align in a temporary pro-regenerative phenotype. FIG. 4B shows that while spontaneous recovery may occur in less severe trauma, surgical reconstruction is useful for functional recovery in severe cases with a defect. In this prolonged period without axonal contact, degradation of the distal pro-regenerative environment and muscle atrophy may diminish potential for recovery. FIG. 4C shows that following delayed nerve repair, functional recovery depends on the location from the injury site (i.e., proximal muscles may have greater recovery than distal muscles). In cases of proximal nerve injury, functional recovery may be achieved in muscles with a shorter regenerative distance than a distal muscle target.

FIG. 5 shows examples of current clinical nerve repair strategies. To date, clinically-available repair strategies focus on facilitating functional recovery by enabling axon regeneration across a defect via bridging strategies, preserving reinnervation capacity via babysitting, or enhancing regeneration via pro-regenerative adjuncts. However, these approaches do not achieve adequate functionality and additional work is useful.

FIG. 6 shows strategies that can be used to enhance nerve regeneration and functional recovery. Current acellular approaches, including biomaterials, extracellular matrix, and growth factors focus on facilitating axonal extension and ignore the importance of the survival of injured neurons and reinnervation capacity of denervated muscle. Pro-regenerative and anti-degenerative biological agents (e.g., small molecules, mRNA, siRNA, and the like) are being assessed for their ability to enhance functional recovery by enhancing regeneration and preventing atrophy. Engineered tissue derived from living scaffolds will likely be needed to achieve greater restoration by replacing the lost nerve tissue with exogenous cells that can communicate with the endogenous nervous system.

FIG. 7 shows examples of tissue engineered medical products (TEMPS) and associated regenerative mechanisms of action. Several TEMPs are in development to address the clinical challenges associated with functional restoration after peripheral nerve injury. For example, genetically-modified xenografts can be utilized for nerve reconstruction without the need for harvesting a donor nerve. Alternatively, recellularized allografts and cell-laden nerve conduits can provide a pro-regenerative niche that promote neuronal health and regeneration. Further advancements will lead to engineered replacement tissue that simultaneously serve as regenerative scaffolds while also providing functional benefits. For example, tissue engineered muscle caps will act as a surrogate end target for regenerating axons, thereby preserving regenerative capacity and preventing painful neuroma formation. Tissue engineered neuromuscular interfaces will be utilized to preserve the reinnervation capacity of denervated muscle with an exogenous source of axons. Tissue engineered nerve grafts will become the gold standard for nerve reconstruction due to their ability to simultaneously preserve regenerative capacity of the proximal neurons and denervated Schwann cells, and maintain the reinnervation potential of denervated muscle.

FIG. 8 shows an example of a surgical algorithm for nerve injury management to maximize ceiling or potential recovery. The timing for nerve repair often depends on the severity of injury. Clinicians will often “wait-and-see” if there is any spontaneous recovery before performing surgery. Although functional recovery often spontaneously occurs following crush injuries, more severe transection injuries require surgery to regain functionality. However, starting almost immediately after injury, the regenerative capacity of the injured proximal neurons diminishes without an appropriate end target and the reinnervation potential of denervated muscle reduces without axonal contact. To address these issues, proximal babysitter grafts comprising exogenous muscle/sensory tissue as a surrogate end target and distal babysitter grafts comprising exogenous neurons as a surrogate axonal source can be used as an early intervention. Delayed repairs can be subsequently performed using a bridging graft at a later time point following removal of the babysitter grafts.

FIG. 9 shows clinical implementation of nerve fusion. Several clinical studies are underway testing the efficacy of nerve fusion. Although the exact mechanism remains unclear, it is believed that the direct reconnection of denucleated distal axons with proximal axons mitigates the harmful effects of prolonged denervation by rapidly restoring connectivity with the disconnected target muscles and/or sensory end organs. Although acute fusion is currently feasible, delayed fusion remains challenging due to catastrophic axonal fragmentation following Wallerian degeneration approximately 3-7 days after injury. To address this issue, tissue engineered neuromuscular interfaces (TE-NMI) can be deployed in the distal nerve to repopulate the denervated nerve sheath with exogenous axons. After the exogenous axons integrate with the denervated muscle/sensory end organ (based on clinical electrophysiological findings), the delayed fusion procedure will be completed during a second surgery that connects proximal axons with denucleated TE-NMI axons. Similar to traditional repairs, tensionless coaptations will likely result in improved clinical recovery. However, TE-NMI axon denucleation will create a non-trivial segmental defect. Therefore, axon-containing nerve grafts can be utilized for fusion across segmental defects greater than 5 mm long.

FIGS. 10A-D show that boldine preserves axonal integrity in an ex vivo sciatic nerve explant model. FIG. 10A shows a schematic of the rat sciatic nerve explant model. Sciatic nerves were harvested, cultured ex vivo with daily media changes, and fixed 3 days after transection. FIG. 10B shows the experimental treatment groups including vehicle control (DMSO) and boldine obtained from two independent vendors. FIG. 10C shows representative immunofluorescence images of axons labeled with neurofilament light chain (NFL-555) at 3 days post-explant. Scale bar: 50 μm. FIG. 10D shows quantification of neurofilament-positive area normalized to total tissue area. Data represent mean±SEM (n=3 explants per group). Vehicle-treated explants exhibited 2.38±0.37% neurofilament-positive area, whereas treatment with Boldine A (Sigma) increased preservation to 5.77±0.84% and Boldine B (Kimum) to 5.52±0.49%. Both boldine preparations showed significantly greater axonal preservation compared with vehicle control (Boldine A vs DMSO, p=0.017; Boldine B vs DMSO, p=0.0237), with no difference between boldine sources (p=0.9539).

FIGS. 11A-E show sustained axon preservation following boldine treatment. FIG. 11A shows an experimental schematic of sciatic nerve explants cultured for 7 days following transection and treated with boldine (10 μM or 100 μM) or vehicle control with daily media replacement. FIG. 11B shows representative low-magnification images of NFL-555-labeled axons at day 7. FIGS. 11C-E show high-magnification insets corresponding to boxed regions in FIG. B. Boldine-treated explants retain more continuous neurofilament labeling compared with DMSO-treated controls, which exhibit fragmented axonal structures consistent with progressive degeneration.

FIGS. 12A-C show SARM1 activity and boldine inhibition using the Transcreener ADPR FP Assay. FIG. 12A shows SARM1 enzyme titration measured using the Transcreener ADPR fluorescence polarization (FP) assay. Increasing enzyme concentration produced a dose-dependent FP signal change with an EC50 of approximately 2.5 nM. Titration experiments were used to identify assay conditions that maintained reactions within the initial velocity range (<20% substrate conversion). FIG. 12B shows ADPR (adenosine 5′-diphosphoribose) standard curve used to convert FP signal to product concentration, enabling quantitative measurement of SARM1-mediated NAD⁺ hydrolysis. FIG. 12C shows dose-response analysis of boldine inhibition of SARM1 NADase activity using 40 nM SARM1. Nonlinear regression analysis yielded an IC50 of approximately 7.5 μM.

FIGS. 13A-C show structural models of SARM1 visualized using UCSF Chimera. FIG. 13A shows octameric assembly of the autoinhibited SARM1 complex (PDB ID: 7CM6). Individual protomers are colored to illustrate quaternary organization. FIG. 13B shows a side-view representation highlighting the stacked architecture of the ARM regulatory, SAM oligomerization, and TIR catalytic domains. FIG. 13C shows the surface representation of the ARM regulatory region showing surface-accessible pockets that may accommodate small-molecule ligands.

FIGS. 14A-B show docking predictions for boldine binding to inactive SARM1. FIG. 14A shows the predicted docking pose of boldine within the autoinhibited SARM1 structure (PDB ID: 7CM6). FIG. 14B shows comparative docking metrics for boldine and reference compounds. Predicted binding affinities (Kd) were estimated from calculated free energies (AG) obtained from docking simulations. Docking procedures and scoring functions are described in the Example 2.

FIGS. 15A-B show docking predictions for boldine binding to activated SARM1. FIG. 15A shows the predicted docking pose of boldine within the activated full-length SARM1 structure (PDB ID: 7NAL). FIG. 15B shows comparative docking metrics for boldine and reference compounds derived from calculated AG values.

FIGS. 16A-B show predicted binding of boldine to the SARM1 ARM regulatory domain. FIG. 16A shows the docking pose of boldine within the isolated ARM regulatory domain structure (PDB ID: 7M6K). FIG. 16B shows comparative docking metrics for boldine and reference compounds, with predicted dissociation constants derived from calculated AG values.

FIGS. 17A-B show predicted binding of boldine to the catalytic TIR domain of SARM1. FIG. 17A shows the predicted docking pose of boldine within the catalytic TIR domain (PDB ID: 6O0Q). FIG. 17B shows comparative docking metrics for boldine and reference compounds derived from docking simulations.

### DETAILED DESCRIPTION

The disclosed methods and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “an”, and “the” include plural reference unless the context clearly dictates otherwise.

“Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list.

As used herein, the term “another” means at least a second or more.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. In some aspects, the subject can be a male. In some aspects, the subject can be a female. In some aspects, the term does not denote a particular sex. Thus, adult, child, adolescent and newborn subjects, as well as fetuses, whether male or female, are intended to be covered. As used herein, the term “subject” refers to either a human or a non-human animal, such as primates, mammals, and vertebrates in need of treatment. In some aspects, the subject in need will or is predicted to benefit from treatment (e.g., treating a neurological nerve injury or a neurodegenerative disease).

As used herein, the term “patient” refers to a subject afflicted with a disease or disorder or neurological nerve injury. The term “patient” includes human and veterinary subjects. In some aspects of the disclosed methods, the “patient” has been diagnosed with a need for treatment for treating a neurological nerve injury or a neurodegenerative disease, such as, for example, prior to the administering step.

As used herein, the term “treat,” “treatment,” or “treating” refers to administration or application of a therapeutic agent to a subject in need thereof, or performance of a procedure or modality on a subject, for the purpose of obtaining at least one positive therapeutic effect or benefit, such as treating a condition, disease or health-related condition. For example, a treatment can include administration of a pharmaceutically effective amount of one or more SARM1 inhibitors, or a composition or formulation thereof that specifically comprises one or more SARM1 inhibitors for the purpose of treating or preventing axonal degeneration or enhancing axonal fusion. The terms “treatment regimen,” “dosing regimen,” or “dosing protocol,” are used interchangeably and refer to the timing and dose of a therapeutic agent, such as one or more SARM1 inhibitors as described herein.

As used herein, the term “administer” or “administration” refers to the act of physically delivering, e.g., via injection or an oral route, a substance as it exists outside the body into a patient, such as by oral, subcutaneous, mucosal, intradermal, intravenous, intramuscular delivery and/or any other method of physical delivery described herein or known in the art. When a disease, disorder or condition, or a symptom thereof, is being treated therapeutically, administration of the substance typically occurs after the onset of the disease, disorder or condition or symptoms thereof. Prophylactic treatment involves the administration of the substance at a time prior to the onset of the disease, disorder or condition or symptoms thereof.

As used herein, the term “contacting” can refer to the placement in direct physical association; includes both in solid and liquid form. “Contacting” is often used interchangeably with “exposed.” In some aspects, “contacting” refers to delivering or exposing a cell (e.g., an axon or a neural tissue) to a molecule (such as a SAMR1 inhibitor and/or a fusogen).

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also be related to a distemper, ailing, ailment, disorder, sickness, illness, complaint, affection. In some aspects, the disease or disorder or condition can be neurological nerve injury or a neurodegenerative disease.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

This present disclosure relates to methods and compositions for controlled intervention in active axonal degeneration processes through targeted SARM1 pathway inhibition and promotion of axonal fusion. The compositions and methods disclosed herein leverage the recent understanding of axon degeneration as an active, enzymatically-driven process rather than passive decay, allowing precise temporal and spatial control of therapeutic intervention that target, for example, SAMR1.

Also disclosed herein are methods for enhanced delivery of SARM1 pathway inhibitors, including but not limited to plant-derived compounds, synthetic molecules, and nucleic acid-based therapeutics, combined with targeting molecules that facilitate retrograde and trans-synaptic transport. Further disclosed herein are manufacturing methods, including but not limited to plant-based production of targeting molecules and conjugates, quality control processes, and scalable production systems.

The compositions and methods disclosed herein can be used for treating peripheral nerve injuries, spinal cord injuries, neurodegenerative diseases (e.g., amyotrophic lateral sclerosis (ALS), Alzheimer's disease, Parkinson's disease, and Multiple Sclerosis (MS)), optic nerve damage, traumatic brain injury, stroke, organ and limb transplantation, engineered tissue transplantation, muscle trauma resulting in axonal degradation, nerve compression or transection, diabetic neuropathy, chemotherapy-induced neuropathy, allograft preservation, and diagnostic evaluation of nerve injuries, including but not limited to iatrogenic injuries and high-risk surgeries.

The compositions and methods disclosed herein address a fundamental challenge in neurological medicine by preventing degeneration of anucleated axons after injury. The compositions and methods disclosed herein can be applicable for the following inter-related applications. When axons are damaged (e.g., physically via stretch or compression, or metabolically via hypoxia), current medical approaches often accept degeneration as inevitable, leading to loss of function and difficult recovery. This approach challenges this paradigm by targeting the active, enzymatic SARM1 pathway that drives axon destruction, thus preventing and/or delaying axon degeneration. However, even after axon transection injuries, the preservation of damaged axons, and in particular, the distal segments separated from their cell bodies (anucleated), maintains residual architecture that facilitates subsequent regeneration and recovery in several ways. First, it supports pro-regenerative Schwann cells that guide new axonal growth and extends the functional window of these Schwann cells to support such axonal re-growth. Second, it prevents deterioration of end target receptors, such as acetylcholine receptors in muscle tissue, which rapidly degrade after denervation, and may aid in preventing catastrophic muscle atrophy. Third, it permits axon-facilitated axonal regeneration, where preserved axonal structures serve as scaffolds and sources of regenerative cues for new axon re-growth. Fourth, preserving distal axons allows for effective neuromodulation of end targets, which has historically suffered poor outcomes due to lack of sustained neurogenic input. Finally, by preventing complete degeneration, this approach can reduce damage signals that can contribute to neuropathic pain and other maladaptive responses. Collectively, delaying degeneration of transected axonal segments may “babysit” distal structures and regenerative pathways, and thereby accelerate regeneration and increase the ceiling for attainable levels of functional recovery. Complementing this preservation strategy, physiological adjuncts were developed to stabilize axons following fusion (i.e., direct reconnection of transection axons) procedures. These adjuncts target calcium homeostasis, mitochondrial support, and cytoskeletal stability-factors in maintaining axonal integrity during the vulnerable post-fusion period. The combination of degeneration prevention with fusion stabilization creates a comprehensive approach to axonal rescue and reconnection. This method can preserve weakly fused axons, allowing faster and greater functional recovery following fusion procedures. This preservation also makes possible delayed nerve fusion, expanding treatment indications well beyond the current restrictive 0-3 day post-injury window that severely limits clinical application.

Also disclosed herein are routes of administration designed to maximize therapeutic efficacy while minimizing systemic exposure. These include but are not limited to oral formulations for systemic delivery, local application methods for direct intervention, slow-release systems for sustained therapeutic levels, and an intramuscular ganglioside-mediated delivery approach. The intramuscular ganglioside-mediated delivery approach utilizes the axon's own retrograde transport machinery to deliver therapeutics from muscle end-plates back to cell bodies, offering targeting precision for axonal preservation.

Nerve regenerative processes are complex, and improvements in functional recovery following major peripheral nerve injury (PNI) are limited by the interplay of at least four factors.

Regenerative Capacity of Proximal Neurons: Injured neurons need sustained support to maintain a regenerative phenotype throughout the useful period of regeneration, which can last weeks to years. Without adequate neurotrophic and/or structural support, the regenerative capacity of the proximal neurons diminishes, leading to reduced axonal growth, potential neuronal loss, and decreased spinal plasticity, collectively reducing the total ceiling for functional recovery.

Ability of Axons and Support Cells to Span the Injury Zone: Axons in peripheral nerve have the potential for spontaneous regeneration and reinnervation given the presence of a suitable substrate connecting the point of injury to the distal end target(s). For less severe injuries, axons use the existing distal nerve architecture, while more severe injuries featuring a segmental defect require surgical repair to replace the missing nervous tissue with suitable grafting material. Autografts are the gold standard for bridging segmental defects, as the donor nerve serves as a living scaffold that replaces the missing segment with similar architecture, rich with autologous pro-regenerative Schwann cells. Levels of functional recovery are strongly influenced by the overall rates and durability of axon re-growth over time.

Capacity of Schwann Cells to Sustain the Distal Pro-Regenerative Environment: Denervated Schwann cells aid regenerating axons by rapidly entering a pro-regenerative state that provides neurotrophic and structural support important for sustaining injured proximal neurons and facilitating muscle reinnervation. However, without axonal contact, the pro-regenerative environment orchestrated by the distal Schwann cells eventually degrades—primarily due to phenotypic changes and eventually cell death of the Schwann cells, halting axonal re-growth and severely reducing functional recovery.

Receptiveness of End Targets for Reinnervation: Severe muscle atrophy swiftly occurs following axonal degeneration. Chronically denervated muscle also exhibits a decreased receptiveness for reinnervation. Indeed, while axons may regenerate to target muscle and attempt to reinnervate, prolonged denervation can result in an inability to form neuromuscular synapses, incomplete synaptic formation, and/or aberrant remodeling at the neuromuscular junction, thus causing diminished functional recovery.

The limitations of peripheral nerve regeneration following severe injury persist despite state-of-the-art surgical interventions, include but not limited to direct neurorrhaphy, nerve conduit/graft implementation, and strategic nerve transfer procedures. While contemporary clinical approaches demonstrate sophistication in addressing fundamental anatomical requirements, the therapeutic scope remains constrained to two primary mechanisms: (1) geometric guidance of regenerating axons across the lesion site, and (2) rudimentary scaffold-based support for axonal extension. Although these interventions facilitate axonal regeneration across defects of limited magnitude (<3 cm), they fail to address the complex cascade of cellular and molecular events that determine functional recovery.

Contemporary clinical strategies demonstrate particular inadequacy in addressing three pathophysiological processes: progressive deterioration of end-organ viability during extended denervation; temporal degradation of Schwann cell pro-regenerative phenotype in the distal nerve segment; and compromised neuronal soma health proximal to the injury zone.

This therapeutic insufficiency assumes heightened significance given the inexorable progression of end-organ denervation and Schwann cell dedifferentiation. The temporal constraints imposed by these degenerative processes-characterized by significant muscle fiber atrophy onset at 3 months post-injury and diminishing Schwann cell supportive capacity by 6 months-establish a therapeutic window after which conventional interventions provide inadequate mechanistic support. This underscores the imperative need for developing next-generation therapeutic strategies that transcend basic geometric guidance to address the intricate cellular and molecular determinants of successful peripheral nerve regeneration.

Peripheral nerve injury is a significant clinical challenge, often leading to permanent functional deficits. Standard interventions, such as autologous nerve grafts or distal nerve transfers, require sacrificing healthy nerve tissue and typically result in limited motor or sensory recovery. Nerve regeneration is complex and influenced by several factors: (1) the regenerative capacity of proximal neurons, (2) the ability of axons and support cells to bridge the injury, (3) the capacity of Schwann cells to maintain a supportive environment, and (4) the readiness of target muscles or sensory organs for reinnervation. Emerging bioengineering solutions, including biomaterials, drug delivery systems, fusogens, electrical stimulation devices, and tissue-engineered products, aim to address these challenges. Effective translation of these therapies requires a deep understanding of the physiology and pathology of nerve injury. Described herein are compositions and methods for developing restorative strategies that address the major physiological responses in nerve repair. By using the compositions and methods described herein can lead to full functional recovery for patients where current approaches offer minimal hope.

### Compositions

Disclosed herein are compositions comprising a sterile alpha and TIR motif-containing protein 1 (SARM1) inhibitor.

Disclosed herein are compositions comprising a sterile alpha and TIR motif-containing protein 1 (SARM1) inhibitor and a biomaterial matrix. In some aspects, the biomaterial matrix can be a hydrogel, a fibrin matrix, a collagen scaffold, a polymer scaffold, or an extracellular matrix formulation. In some aspects, wherein the composition can be configured for local delivery to neural tissue.

In some aspects, the SARM1 inhibitor can be an isoquinoline alkaloid, an isothiazole, a benzisothiazole, or an isothiazolinone derivative. In some aspects, the isoquinoline alkaloid can be boldine. In some aspects, the SARM1 inhibitor can be dehydronitrosonisodipine (dHNN) or DSRM-3716.

Any of the compositions disclosed herein can be formulated or configured for local delivery to neural tissue. Any of the compositions disclosed herein can be formulated or configured for local delivery to one or more axons.

The compositions described herein can be formulated to include a therapeutically effective or an effective amount of a SARM1 inhibitor described herein. Therapeutic administration encompasses prophylactic applications (e.g., or preventing degradation). Based on genetic testing and other prognostic methods, a physician in consultation with their patient can choose a prophylactic administration where the patient has a clinically determined predisposition or increased susceptibility (in some cases, a greatly increased susceptibility) to axonal degradation.

The compositions described herein can be administered to the subject (e.g., a human patient) in an amount sufficient to delay, reduce, or preferably prevent the onset of clinical disease (e.g., degradation of one or more axons). Accordingly, in some aspects, the patient can be a human patient. In therapeutic applications, compositions can be administered to a subject (e.g., a human patient) already with or diagnosed with a neurological nerve injury (e.g., severed axon, or axon injury; spinal cord injury, peripheral nerve injury, optic nerve injury) or a neurodegenerative disease (e.g., Parkinson's disease, Alzheimer's disease, amyotrophic lateral sclerosis (ALS), multiple sclerosis, glaucoma, optic neuropathy, chemotherapy-induced neuropathy, or peripheral neuropathy) in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease, disorder, condition or injury is delayed, hindered, or prevented, or the disease, disorder, condition or injury or a symptom of the disease, disorder, condition or injury is ameliorated or its frequency can be reduced. One or more of the symptoms can be less severe. Recovery can be accelerated in an individual who has been treated. For example, treatment of a neurological nerve injury or a neurodegenerative disease may involve, for example, enhancing fusion of one or more axons, delaying degeneration of the fused axons, delaying Wallerian degeneration in fused axons, reducing or delaying degeneration in an axon, reducing or delaying degeneration in a neural tissue, inhibiting SARM1 NADase activity or activation, preventing degeneration in the injured axon, transplanting fused axon(s), or preserving axonal integrity within an engineered neural tissue construct.

In some aspects, the SARM1 inhibitor can be administered with at least a second therapeutic agent. The methods and compositions, including combination therapies, can enhance the therapeutic or protective effect, and/or increase the therapeutic effect of any of the SARM1 inhibitors described herein.

The SARM1 inhibitors can be administered before, during, after, or in various combinations relative to a second therapeutic agent or therapy. The administrations may be in intervals ranging from concurrently to minutes to days to weeks. In aspects where the SARM1 inhibitor is provided to a patient separately from a second therapeutic agent or therapy, one would generally ensure that a significant period of time did not expire between the time of each delivery, such that the two compounds would still be able to exert an advantageously combined effect on the patient. In such instances, it is contemplated that one may provide a patient with the SARM1 inhibitor and the second therapeutic agent or therapy within about 1 second to 1 minute or 1 second to one hour of each other.

In some aspects, a course of treatment can last between 1-14 days or more (this such range includes intervening days). It is contemplated that one agent may be given on any day of day 1 to day 14 (this such range includes intervening days) or any combination thereof, and another agent is given on any day of day 1 to day 14 (this such range includes intervening days) or any combination thereof. Within a single day (24-hour period), the patient may be given one or multiple administrations of the agent(s). In some aspects, the treatment cycles can be repeated as necessary.

Various combinations may be employed. For the example below a SARM1 inhibitor is designated as “A” and a second therapeutic agent is designated as “B”:

A/B/A, B/A/B, B/B/A, A/A/B, A/B/B, B/A/A, A/B/B/B, B/A/B/B, B/B/B/A, B/B/A/B,
A/A/B/B, A/B/A/B, A/B/B/A, B/B/A/A, B/A/B/A, B/A/A/B, A/A/A/B, B/A/A/A, A/B/A/A, or
A/A/B/A.

Administration of any compound or therapy disclosed herein to a patient will follow general protocols for the administration of such compounds, taking into account the toxicity, if any, of the agents. Therefore, in some aspects there can be a step of monitoring toxicity that can be attributable to combination therapy.

In some aspects, the second therapeutic agent can be a fusogen. Examples of a fusogen include but are not limited to can be a polyethylene glycol (PEG), chitosan, a fusogenic lipid, a membrane fusion peptide, or a viral fusogen.

In any of the methods disclosed herein, contact or administration of a SARM1 inhibitor alone or with a fusogen can be followed by transplantation of the treated neural tissue or fused axons.

The compositions described herein used in the disclosed methods can be formulated to include a therapeutically effective amount of the SARM1 inhibitor disclosed herein. In some aspects, the SARM1 inhibitor thereof disclosed herein can be contained within a pharmaceutical formulation. In some aspects, the pharmaceutical formulation can be a unit dosage formulation. In some aspects, the pharmaceutical formulation can be in a biomaterial matrix. In some aspects, the biomaterial matrix can be a hydrogel, a polymer scaffold, a fibrin matrix, a collagen matrix, or a synthetic extracellular matrix material.

The therapeutically effective amount or dosage of any of the SARM1 inhibitors used in the methods as disclosed herein applied to mammals (e.g., humans) can be determined by one of ordinary skill in the art with consideration of individual differences in age, weight, sex, the severity of the subject's symptoms or injury, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the symptoms), the age and physical characteristics of the subject and other considerations known to those of ordinary skill in the art. Dosages can be established using clinical approaches known to one of ordinary skill in the art. A therapeutically effective dosage of the SARM1 inhibitor can result in a decrease in severity of one or more disease symptoms, an increase in frequency and duration of disease symptom-free periods, or a prevention of impairment or disability due to the disease affliction. As disclosed therein, in some aspects a therapeutically effective amount of a SARM1 inhibitor can reduce or delay degeneration in one or more axons or neural tissue, prevent degeneration of an injured axon, enhance fusion of one or more severed axons, delay degeneration of the fused axons, delay Wallerian degeneration in one or more axons or neural tissue, permit transmission of electrical signals across the fused axons following implantation, or preserve axonal integrity within an engineered neural tissue construct, and preservation of axonal integrity or otherwise reduce or ameliorate one or more symptoms in a subject.

The duration of treatment with a SARM1 inhibitor in the methods disclosed herein can be any length of time from as short as one second to as long 14 days or longer. For example, the compositions can be applied or in contact with the injured neuron, injured axon, or neural tissue for at least 1 minute, 5 minutes, 10 minutes, 15 minutes, 20 minutes, 25 minutes, 30 minutes, 35 minutes, 40 minutes, 45 minutes, 50 minutes, 55 minutes, 60 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 2 days, 3 days, 4 days, 5 days, 6 days, 7 days, 8 days, 9 days, 10 days, 11 days, 12 days, 13 days, 14 days, or longer prior to a transplantation or implantation of the neural tissue. In some aspects, the neural tissue can be maintained ex vivo in the presence of the SARM1 inhibitor for a period of 1 minute to 14 days prior to transplantation. In some aspects, the contacting step can occur for a period of at least 1 hour. In some aspects, the contacting step can occur for a period of at least 6 hours. In some aspects, the contacting step can occur for a period of at least 24 hours. In some aspects, the contacting step can occur for a period of at least 72 hours. In some aspects, the contacting step can occur for continuously for 1 or more days. In some aspects, the contacting step can occur for continuously for 2 or more days. In some aspects, the contacting step can occur for continuously for 3 or more days. In some aspects, the contacting step can occur for continuously for 4 or more days. In some aspects, the contacting step can occur for a period 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42, hours, 48, hours, 72 hours, or any amount of time in between. In some aspects, the contacting step can occur for a period 1 day, 2 days, 3 days, 4 days, 5 days, 6 days, 7 days, or longer. In some aspects, the composition in the methods disclosed herein as short as one day or for the length of the life span of the host. In some aspects, the SARM1 inhibitor can be administered once a day, once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the SARM1 inhibitor as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure. In some aspects, the SARM1 inhibitor can be administered to the subject intravenously, intramuscularly, intrathecally, orally, intraperitoneally, locally to a nerve injury site, or via sustained-release delivery. In some aspects, boldine can be administered to a subject in need thereof at a dose of 0.001 mg/kg to 10 mg/kg.

The duration of treatment with a fusogen in the methods disclosed herein can be any length of time from as short as one second to as long 30 minutes or longer. For example, the compositions can be applied or in contact with the injured neuron, injured axon, or neural tissue for at least 1 second, 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, 50 seconds, 51 seconds, 52 seconds, 53 seconds, 54 seconds, 55 seconds, 56 seconds, 57 seconds, 58 seconds, 59 seconds, 60 seconds or longer. In some aspects, the fusogen can contact the neural tissue for 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes. In some aspects, the neural tissue can be maintained ex vivo in the presence of the SARM1 inhibitor for a period of 1 minute to 14 days prior to transplantation. In some aspects, the contacting step can occur for a period of at least 30 minutes. It is also noted that the frequency of treatment can be variable. For example, the present compositions can be administered once (or twice, three times, etc.) hourly, daily, or weekly.

The total effective amount of the fusogen as disclosed herein can be administered to a subject as a single dose, either as a bolus or by infusion over a relatively short period of time, or can be administered using a fractionated treatment protocol in which multiple doses are administered over a more prolonged period of time. In some aspects, the fusogen can be administered to the neural tissue or severed axon locally to a nerve injury site or via sustained-release delivery.

### Methods

Disclosed herein are methods of inhibiting sterile alpha and TIR motif-containing protein 1 (SARM1) with a SARM1 inhibitor. Also disclosed herein are methods of inhibiting NADase with a SARM1 inhibitor. Further disclosed herein are methods of modulating SARM1 activity. In some aspects, the methods can comprise administering boldine. In some aspects, boldine binds within the NAD⁺ catalytic site of SARM1 and inhibits SARM1 activation. Also disclosed herein are methods of inhibiting SARM1 activation. In some aspects, the methods can comprise administering boldine in an amount sufficient to interfere with TIR domain multimerization and downstream NADase activation.

Disclosed herein are methods of inhibiting sterile alpha and TIR motif-containing protein 1 (SARM1) NADase activity in an axon. In some aspects, the methods can comprise: contacting the axon with an effective amount of a SARM1 inhibitor, thereby inhibiting SARM1 NADase activity in the axon. In some aspects, the contacting step can occur for a period of at least 1 hour. In some aspects, the contacting step can occur for a period of at least 6 hours. In some aspects, the contacting step can occur for a period of at least 24 hours. In some aspects, the contacting step can occur for a period of at least 72 hours. In some aspects, the contacting step can occur for continuously for 1 or more days. In some aspects, the contacting step can occur for a period 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42, hours, 48, hours, 72 hours, or any amount of time in between. In some aspects, the contacting step can occur for a period 1 day, 2 days, 3 days, 4 days, 5 days, 6 days, 7 days, or longer. In some aspects, the SARM1 inhibitor can be selected from the group consisting of an isoquinoline alkaloid, an isothiazole, a benzisothiazole, and an isothiazolinone derivative. In some aspects, the isoquinoline alkaloid can be boldine. In some aspects, the SARM1 inhibitor can be dehydronitrosonisodipine (dHNN) or DSRM-3716. In some aspects, the contacting step can be performed ex vivo, in vitro, in situ, or in vivo. In some aspects, in vivo can be in a subject. In some aspects, in vivo can be in a human subject. In some aspects, the contacting step can be by local or regionally-restricted delivery of the SARM1 inhibitor to the axon. In some aspects, the local delivery or the regionally-restricted delivery can be with a slow release hydrogel, polymer scaffold, osmotic pump, catheter, injectable depot, or controlled-release formulation. In some aspects, the SARM1 inhibitor can be delivered locally to the axon at a site of injury. In some aspects, boldine inhibits SARM1 NADase activity by binding to a catalytic domain of SARM1 or regulatory domain of SARM1. In some aspects, boldine preferentially binds to an autoinhibited conformation or structure of SARM1, thereby stabilizing the autoinhibited conformation or structure and inhibiting SARM1 activation or inhibiting SARM1 activity. In some aspects, stabilization of the autoinhibited conformation or structure can reduce multimerization of TIR catalytic domains. TIR catalytic domains are needed for NADase activity.

Disclosed herein are methods of explant therapeutic conditioning. Also disclosed herein are methods of preserving axonal integrity in an ex vivo neural tissue explant. In some aspects, the methods can comprise contacting the explant with an effective amount of a sterile alpha and TIR motif-containing protein 1 (SARM1) inhibitor (e.g., boldine) in an amount sufficient to reduce structural axon degeneration relative to untreated control.

Disclosed herein are methods of reducing or delaying degeneration in an axon. In some aspects, the methods can comprise: contacting the axon with an effective amount of a sterile alpha and TIR motif-containing protein 1 (SARM1) inhibitor, thereby reducing or delaying degeneration of the axon. In some aspects, the SARM1 inhibitor can be selected from the group consisting of an isoquinoline alkaloid, an isothiazole, a benzisothiazole, and an isothiazolinone derivative. In some aspects, the isoquinoline alkaloid can be boldine. In some aspects, the SARM1 inhibitor can be dehydronitrosonisodipine (dHNN) or DSRM-3716. In some aspects, the SARM1 inhibitor an isothiazole compound. In some aspect, isothiazole compound can be a benzisothiazole derivative, an isothiazolinone derivative, or a substituted isothiazole compound. In some aspects, the SARM1 inhibitor can be boldine. In some aspects, boldine can inhibit SARM1 NADase activity by binding to a catalytic site or domain of SARM1 or regulatory domain of SARM1, thereby inhibiting SARM1 activation. In some aspects, the methods can further comprise quantifying axonal degeneration by measuring neurofilament-positive area within neural tissue. Axonal preservation or reductions or delays in degeneration of an axon can be quantified using neurofilament immunofluorescence as a marker of intact axons. Degeneration results in fragmented labeling, while preserved axons retain continuous signal. In some aspects, quantification reducing or delaying degeneration in an axon can be performed by measuring the percentage of neurofilament-positive area relative to total tissue area. In some aspects, the neurofilament-positive area can comprises a neurofilament protein. In some aspects, the neurofilament protein can comprise a neurofilament light chain (NF-L). In some aspects, the reduction in axon degeneration can be determined by comparing the axon contacted with the SARM1 with an axon not contacted with the SARM1 inhibitor. In some aspects, the reduction in axon degeneration can be reduced in the axon contacted with the SARM1 inhibitor compared to an axon not contacted with the SARM1 inhibitor. In some aspects, the reduction in axon degeneration can be maintained for at least 3 days. In some aspects, the reduction in axon degeneration can be maintained for at least 7 days. In some aspects, the reduction in axon degeneration can be maintained for at least 1 day, 2 days, 3 days, 4 days, 5 days, 6 days, 7 days, or longer. In some aspects, the contacting step can be performed ex vivo on a neural tissue explant comprising the axon.

Disclosed herein are methods of explant conditioning for transplantation. Disclosed herein are methods of treating or preventing axonal degeneration associated with neurological injury or neurodegenerative disease. In some aspects, the methods can comprise administering an effective amount of a sterile alpha and TIR motif-containing protein 1 (SARM1) inhibitor (e.g., boldine) to neural tissue ex vivo prior to transplantation or implantation into a subject. In some aspects, the neural tissue can comprise a nerve graft, a spinal cord tissue, an optic nerve tissue, or an engineered neural tissue.

Disclosed herein are methods of treating or preventing degeneration of an injured axon. In some aspects, the methods can comprise: contacting a neural tissue comprising the axon ex vivo with an effective amount of a sterile alpha and TIR motif-containing protein 1 (SARM1) inhibitor, thereby treating or preventing degeneration in the injured axon. In some aspects, the neural tissue can be a nerve graft, a spinal cord tissue, an optic nerve tissue or an engineered neural tissue. In some aspects, the contacting step can be performed prior to a transplantation or implantation of the neural tissue. In some aspects, the contacting step can be performed 1 minute, 5 minutes, 10 minutes, 15 minutes, 20 minutes, 25 minutes, 30 minutes, 35 minutes, 40 minutes, 45 minutes, 50 minutes, 55 minutes, 60 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 2 days, 3 days, 4 days, 5 days, 6 days, 7 days, 8 days, 9 days, 10 days, 11 days, 12 days, 13 days, 14 days, or longer prior to a transplantation or implantation of the neural tissue. In some aspects, the methods can further comprise transplanting or implanting the neural tissue into a subject. In some aspects, the neural tissue can be maintained ex vivo in the presence of the SARM1 inhibitor for a period of 1 minute to 14 days prior to transplantation. In some aspects, the neural tissue can be determined to be ready for transplantation by measuring neurofilament-positive area or axonal continuity within the tissue. In some aspects, the subject has a neurological injury or a neurodegenerative disease. In some aspects, the neurodegenerative disease can be Parkinson's disease, Alzheimer's disease, amyotrophic lateral sclerosis (ALS), multiple sclerosis, glaucoma, optic neuropathy, chemotherapy-induced neuropathy, or peripheral neuropathy. In some aspects, the neurological injury can be a peripheral nerve injury, a spinal cord injury, or an optic nerve injury. In some aspects, the contacting step can occur for a period of at least 1 hour. In some aspects, the contacting step can occur for a period of at least 6 hours. In some aspects, the contacting step can occur for a period of at least 24 hours. In some aspects, the contacting step can occur for a period of at least 72 hours. In some aspects, the contacting step can occur for continuously for 1 or more days. In some aspects, the contacting step can occur for a period 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42, hours, 48, hours, 72 hours, or any amount of time in between. In some aspects, the contacting step can occur for a period 1 day, 2 days, 3 days, 4 days, 5 days, 6 days, 7 days, or longer. In some aspects, the SARM1 inhibitor can be selected from the group consisting of an isoquinoline alkaloid, an isothiazole, a benzisothiazole, and an isothiazolinone derivative. In some aspects, the isoquinoline alkaloid can be boldine. In some aspects, the SARM1 inhibitor can be dehydronitrosonisodipine (dHNN) or DSRM-3716. In some aspects, the SARM1 inhibitor can be formulated for sustained release. In some aspects, the SARM1 inhibitor is in a biomaterial matrix. In some aspects, the biomaterial matrix can be a hydrogel, a polymer scaffold, a fibrin matrix, a collagen matrix, or a synthetic extracellular matrix material. In some aspects, the biomaterial matrix can comprise an effective amount of a sterile alpha and TIR motif-containing protein 1 (SARM1) inhibitor. In some aspects, the biomaterial matrix can provide sustained local release of the SARM1 inhibitor. In some aspects, the SARM1 inhibitor can be administered via a hydrogel (e.g., a slow release hydrogel), polymer scaffold, fibrin matrix, collagen matrix, synthetic extracellular matrix material, osmotic pump, catheter, injectable depot, or a controlled-released formulation.

Disclosed herein are methods of administering fusion-promoting therapeutics. Also disclosed herein are methods of extending the amount of time needed for enhancing axon fusion following a neurological injury. Also disclosed herein are methods of enhancing axonal fusion following neurological injury. In some aspects, the methods can comprise: (a) contacting an injured neural tissue ex vivo with an effective amount of a sterile alpha and TIR motif-containing protein 1 (SARM1) inhibitor prior to or concurrent with administration of a fusogen; and (b) administering the fusogen to reconnect severed axons. In some aspects, the methods can comprise: (a) contacting an injured neural tissue ex vivo with boldine prior to or concurrent with administration of a fusogen; and (b) administering the fusogen to reconnect severed axons. In some aspects, the boldine can preserve axonal integrity sufficient to improve functional reconnection following fusion.

Disclosed herein are methods of enhancing fusion of one or more severed axons in a subject in need thereof. In some aspects, the methods comprising: (a) contacting ex vivo a neural tissue comprising the one or more severed axons with an effective amount of a sterile alpha and TIR motif-containing protein 1 (SARM1) inhibitor; and (b) contacting ex vivo the neural tissue comprising the one or more severed axons in (a) with an effective amount of a fusogen, wherein the contacting in (a) is prior to or concurrent with the contacting of (b), thereby enhancing the fusion of the one or more severed axons in the subject. Disclosed herein are methods of delaying degeneration of one or more fused axons. In some aspects, the methods can comprise: (a) contacting ex vivo a neural tissue comprising the one or more fused axons with an effective amount of a sterile alpha and TIR motif-containing protein 1 (SARM1) inhibitor; and (b) contacting ex vivo the neural tissue comprising the one or more fused axons in (a) with an effective amount of a fusogen, wherein the contacting in (a) is prior to or concurrent with the contacting of (b), thereby delaying degeneration of the one or more severed axons. Disclosed herein are methods of enhancing fusion of one or more severed axons and/or delaying degeneration of the subsequently fused axons. In some aspects, the methods can comprise: (a) contacting ex vivo a neural tissue comprising the one or more severed axons with an effective amount of a sterile alpha and TIR motif-containing protein 1 (SARM1) inhibitor; and (b) contacting ex vivo the neural tissue comprising the one or more severed axons in (a) with an effective amount of a fusogen, wherein the contacting in (a) is prior to or concurrent with the contacting of (b), thereby enhancing the fusion of the one or more severed axons and/or delaying degeneration of the subsequently fused axons. In some aspects, the SARM1 inhibitor can delay Wallerian degeneration in the subsequently fused axons. In some aspects, the one or more severed axons can be injured via a neurological nerve injury. In some aspects, the neurological nerve injury can be a peripheral nerve injury, a spinal nerve injury, or an optic nerve injury. In some aspects, the fusogen can be a polyethylene glycol (PEG), chitosan, a fusogenic lipid, a membrane fusion peptide, or a viral fusogen. In some aspects, the fusogen can contact the neural tissue by topical application, perfusion, or microinjection. In some aspects, the fusogen can contact the neural tissue for 1 second to 30 minutes. In some aspects, the fusogen can contact the neural tissue for 1 second, 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, 50 seconds, 51 seconds, 52 seconds, 53 seconds, 54 seconds, 55 seconds, 56 seconds, 57 seconds, 58 seconds, 59 seconds, 60 seconds or longer. In some aspects, the fusogen can contact the neural tissue for 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes. In some aspects, the methods can preserve the integrity of the one or more fused axons or subsequently fused axons thereby permitting transmission of electrical signals across the fused axons following implantation.

Disclosed herein are methods of treating a neurological injury or a neurodegenerative disease using a systemic mechanism-anchored therapeutic. Also disclosed herein are methods of treating a neurological injury or a neurodegenerative disease. In some aspects, the methods can comprise administering an effective amount of a sterile alpha and TIR motif-containing protein 1 (SARM1) inhibitor to a subject in need thereof in an effective amount to inhibit SARM1 pathway activation. In some aspects, the methods can comprise administering boldine to a subject in need thereof in an effective amount to inhibit SARM1 pathway activation.

Disclosed herein are methods of treating a neurological nerve injury or a neurodegenerative disease in a subject in need thereof. In some aspects, the methods can comprise: administering to the subject an effective amount of a sterile alpha and TIR motif-containing protein 1 (SARM1) inhibitor, thereby treating a neurological injury or a neurodegenerative disease in the subject. In some aspects, the neurological nerve injury can be a peripheral nerve injury, a spinal nerve injury, or an optic nerve injury. In some aspects, the neurodegenerative disease can be Parkinson's disease, Alzheimer's disease, amyotrophic lateral sclerosis (ALS), multiple sclerosis, glaucoma, optic neuropathy, chemotherapy-induced neuropathy, or peripheral neuropathy. In some aspects, the SARM1 inhibitor can be selected from the group consisting of an isoquinoline alkaloid, an isothiazole, a benzisothiazole, and an isothiazolinone derivative. In some aspects, the isoquinoline alkaloid can be boldine. In some aspects, the SARM1 inhibitor can be dehydronitrosonisodipine (dHNN) or DSRM-3716. In some aspects, the SARM1 inhibitor an isothiazole compound. In some aspect, isothiazole compound can be a benzisothiazole derivative, an isothiazolinone derivative, or a substituted isothiazole compound. In some aspects, the SARM1 inhibitor can be boldine. In some aspects, the SARM1 inhibitor can be administered intravenously, intramuscularly, intrathecally, orally, intraperitoneally, locally to a nerve injury site, or via sustained-release delivery. In some aspects, boldine can be administered at a dose of 0.001 mg/kg to 10 mg/kg. In some aspects, boldine can inhibit SARM1 NADase activity by binding to a catalytic domain of SARM 1 or regulatory domain of SARM1.

Disclosed herein are methods of using engineered tissue therapeutics.

Disclosed herein are methods of preserving axonal integrity within an engineered neural tissue construct. In some aspects, the methods can comprise contacting the engineered neural tissue construct with an effective amount of a sterile alpha and TIR motif-containing protein 1 (SARM1) inhibitor, thereby preserving axonal integrity axonal continuity within the engineered neural tissue construct prior to implantation into a subject. In some aspects, the methods further maintain axonal continuity with the engineered neural tissue construct prior to implantation into a subject. In some aspects, the engineered neural tissue construct can comprise aligned axonal tracts. In some aspects, the engineered neural tissue construct can comprise neurons derived from stem cells. In some aspects, the engineered neural tissue construct can comprise neurons derived from stem cell-derived spheroids. In some aspects, the engineered neural tissue construct can comprise neurons derived from stem cell-derived organoids. In some aspects, the engineered neural tissue construct can be maintained in culture in the presence of the SARM1 inhibitor for a period of 1 hour to 14 days prior to implantation. In some aspects, the engineered neural tissue construct can be delivered to a subject within a hydrogel or extracellular matrix scaffold, wherein the hydrogel or extracellular matrix scaffold comprises the SARM1 inhibitor. In some aspects, the engineered neural tissue construct can comprise elongated axonal tracts capable of transmitting electrical signals between neuronal populations. In some aspects, the preservation of axonal integrity permits transmission of electrical signals across the engineered neural tissue construct following implantation.

In any of the methods disclosed herein, the axon can be that of a neuron. In some aspects, the axon can be injured. In some aspects, the axon can be at risk of degeneration. In some aspects, the neuron can be selected from the group consisting of a sensory neuron, motor neuron, interneuron, retinal ganglion cell, cortical neuron, spinal neuron, and dorsal root ganglion neuron. In some aspects, the sensory neuron can be a peripheral sensory neuron. In some aspects, the axon can be a central nervous system axon. In some aspects, the axon can be a peripheral nervous system axon.

In some aspects, the SARM1 inhibitor can be administered prophylactically to reduce or prevent axonal degeneration following a neurological injury. In some aspects, the SARM1 inhibitor can be locally applied to the axon at a site of injury.

In any of the methods disclosed herein, the methods can further comprising administering or contacting a neural tissue or axon(s) with a fusogen, a nerve graft, an engineered neural tissue construct, electrical stimulation, one or more neurotrophic factors, or an axonal guidance scaffold. In any of the methods disclosed herein, the methods can further comprise administering or contacting a neural tissue or axon(s) the SARM1 inhibitor in conjunction with one or more of a fusogen, a nerve graft, an engineered neural tissue construct, electrical stimulation, one or more neurotrophic factors, or an axonal guidance scaffold.

### Pharmaceutical Compositions

As disclosed herein, are pharmaceutical compositions, comprising one or more of the therapeutic compositions or SARM1 inhibitors disclosed herein. As disclosed herein, are pharmaceutical compositions, comprising a SARM1 inhibitor and a pharmaceutical acceptable carrier described herein. In some aspects, the SARM1 inhibitor can be formulated for oral or parenteral administration. In some aspects, the parenteral administration can be intravenous, intramuscular, intrathecal, or intraperitoneally. In some aspects, the administration can be direct injection. In some aspects, the administration can be locally to a nerve injury site. In some aspects, the administration can be via sustained-release delivery. In some aspects, the SARM1 inhibitor can be administered intravenously, intramuscularly, intrathecally, orally, intraperitoneally, locally to a nerve injury site, or via sustained-release delivery. In some aspects, the SARM1 inhibitor can be formulated with or within a biomaterial matrix. In some aspects, the biomaterial matrix can be a hydrogel, a fibrin matrix, a collagen scaffold, a polymer scaffold, or an extracellular matrix formulation. In some aspects, the compositions can be formulated for administration via a slow release hydrogel, polymer scaffold, osmotic pump, catheter, injectable depot, or a controlled-released formulation.

The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., biomaterial matrix or osmotic pump) may increase the efficiency of delivery. For example, the SARM1 inhibitor can be administered directly to the injury site or directly to the axon or neural tissue by local delivery or regionally-restricted delivery. In some aspects, the SARM1 inhibitor can be administered via a slow release hydrogel, polymer scaffold, osmotic pump, catheter, injectable depot, or a controlled-released formulation.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient or directly to the axon, neural tissue, or injury site.

The pharmaceutical compositions as disclosed herein can be prepared for oral or parenteral administration. Pharmaceutical compositions prepared for parenteral administration include those prepared for intravenous, intramuscular, subcutaneous, intraperitoneal, intrathecal, transmucosal (e.g., intranasal, intravaginal, or rectal), or transdermal (e.g., topical) administration. Aerosol inhalation can also be used. The compositions can be prepared for directly or local delivery. The compositions can also be prepared for sustained release including biomaterial matrix formulations, hydrogels, polymer scaffolds, osmotic pumps, catheters, injectable depots, or a controlled-release formulation Thus, compositions can be prepared for parenteral administration that includes any of the SARM1 inhibitors dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

### Kits

The composition described herein can be packaged in a suitable container labeled, for example, for use as a therapy for inhibiting SARM1 NADase activity in an axon, reducing or delaying degeneration in an axon, treating or preventing degeneration of an injured axon, enhancing fusion of one or more severed axons, and delaying degeneration of one or more fused axons, treating a neurological nerve injury or a neurodegenerative disease in a subject in need thereof, preserving axonal integrity within an engineered neural tissue construct or any of the methods disclosed herein. Accordingly, packaged products (e.g., sterile containers containing the composition described herein and packaged for storage, shipment, or sale at concentrated or ready-to-use concentrations) and kits, including at least one or more of the SARM1 inhibitors and optionally a fusogen as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compositions can be ready for administration (e.g., present in dose-appropriate units), and may include a pharmaceutically acceptable adjuvant, carrier or other diluent. Alternatively, the compositions can be provided in a concentrated form with a diluent and instructions for dilution.

### Illustrative Embodiments

Embodiment 1: A method for preserving residual functionality and accelerating functional recovery after neurological injury by delaying axon degeneration in a subject, the method comprising: a) Facilitating axon-facilitated axonal regeneration by stabilizing existing axonal structures, enhancing metabolic support, and utilizing regenerative cues from surviving axons; b) Promoting pro-regenerative Schwann cells through targeted transplantation, secretome therapy, or gene modulation to enhance targeted regeneration and reinnervation; or c) Maintaining distal end targets by preventing denervation-induced muscle atrophy, preserving synaptic integrity, and supporting long-term neuromuscular connectivity.

Embodiment 2: The method of embodiment 1, further comprising prolonging axonal survival post-injury to maintain residual functionality by: a) Inhibiting calcium-dependent axonal sealing and neuronal degeneration; b) Administering one or more mitochondrial support agents to directly sustain axonal integrity, wherein the one or more mitochondrial support agents are nicotinamide riboside (NR), nicotinamide mononucleotide (NMN), NAD precursors, or Vitamin B12; c) Regulating chronic calcium homeostasis and oxidative stress by modulating ion channel activity and delivering one or more antioxidants to prevent progressive axon-autonomous degeneration, wherein the one or more antioxidants are boldine or glutathione; d) Utilizing ion channel modulators to stabilize axonal membranes and reduce excitotoxicity; e) Transplanting Schwann cells or delivering Schwann cell-derived extracellular vesicles (secretomes) into the injured nerve environment to enhance axonal support; or f) Delivering trophic factors or engineered Schwann cell secretome therapies to sustain axonal survival and promote regeneration.

Embodiment 3: The method of embodiment 1, further comprising stabilizing muscle endplates post-denervation by: a) Applying electrical stimulation to denervated muscle; or b) Providing localized trophic support to prevent muscle atrophy.

Embodiment 4: A pharmaceutical composition comprising one or more neuroprotective agents (e.g., boldine), NAD precursors, SARM1 inhibitors, or ganglioside-targeted delivery agents, formulated for oral, local, slow-release, or intramuscular administration.

Embodiment 5: A method for delivering ganglioside-based neuroprotectants, the method comprising: a) Formulating a neuroprotectant agent for intramuscular injection; b) permitting axonal uptake via ganglioside endocytosis; or c) promoting neural repair through enhanced intracellular signaling.

Embodiment 6: A method for retrograde axon protection, the method comprising: a) Targeting axonal terminals with one or more SARM1 inhibitors, neurotrophic factors, or metabolic support agents; b) Utilizing GM1-based, CTB-mediated, or other transport mechanisms to permit retrograde transport; or c) Delivering one or more therapeutic agents from distal axonal terminals to the soma.

Embodiment 7: A method for trans-synaptic axon protection, the method comprising: a) Administering one or more neuroprotective agents capable of crossing synapses; b) Utilizing WGA transport systems, targeting peptides, or other synapse-penetrating mechanisms; or c) Facilitating the transfer of neurotrophic or SARM1-inhibitory compounds between connected neuronal populations.

Embodiment 8: The method of embodiment 2, wherein the one or more mitochondrial support agents is boldine, a connexin hemichannel blocker, for enhancing axonal survival.

Embodiment 9: The method of embodiment 2, further comprising administering an agent that promotes the cytoskeletal stabilization, wherein the agent is capable of pharmacological inhibition of SARM1 or administration of a SARM1 siRNA.

Embodiment 10: The method of embodiment 2, further comprising metabolic supplementation, wherein the metabolic supplementation includes but is not limited to delivery of Schwann cell-derived secretome, engineered secretome, or extracellular vesicle-based therapy for enhanced neuroprotection.

Embodiment 11: The method of embodiments 2 and 5, wherein axonal survival is enhanced through a combination therapy approach, wherein the combination therapy approach comprises: a) Co-administration of one or more SARM1 inhibitors with one or more mitochondrial support agents to maximize neuroprotection; b) Combining ganglioside-based targeting with retrograde transport mechanisms for enhanced drug delivery; or c) using antioxidant therapy alongside connexin hemichannel blockers to reduce oxidative stress and prolong axonal integrity.

Embodiment 12: A method for immediate axonal reconnection within 0 days to 1 week post-injury using a fusogen, the method comprising: a) Administering a fusogen, wherein the fusogen is polyethylene glycol (PEG) or chitosan to reconnect severed axons; b) Applying axon protection strategies to promote fused axon survival; or c) Sustaining residual functional benefits and promoting earlier and greater recovery post-injury.

Embodiment 13: A method for delayed axonal reconnection following injury after 1 week post-injury, the method comprising: a) Implementing axon protection strategies to sustain axonal integrity beyond 1 week; b) Utilizing neuroprotectants or metabolic support agents to prevent further degeneration; or c) Applying a fusogen, wherein the fusogen is PEG or chitosan to enable reconnection and functional restoration at delayed timepoints.

Embodiment 14: The method of embodiment 2, wherein the transplanted Schwann cells are genetically modified to overexpress one or more neurotrophic factors, wherein the one or more neurotrophic factors are BDNF, CNTF, or GDNF, to enhance trophic support and improve axon survival.

Embodiment 15: A method for preserving axonal viability for transplantation purposes, the method comprising: a) Preserving donor axons for allograft fusion; b) Maintaining donor organ axons and end targets; or c) Preserving engineered axon tracts for transplantation.

Embodiment 16: The method of embodiment 2, wherein hydrogel microcolumns serve as a scaffold for axonal survival and guided regeneration by supporting Schwann cell migration, trophic factor delivery, and axonal alignment.

Embodiment 17: The method of embodiment 2, wherein hydrogel microcolumns are loaded with an axon protectant to enhance the survival of both the patient's native tissue and the engineered tissue.

Embodiment 18: A method for prophylactic neuroprotection following neurological injury when the severity of the deficit is unknown, the method comprising early administration of axon-protective agents to prevent irreversible degeneration and maintain functional recovery potential.

Embodiment 19: A method for treating peripheral nerve injuries by enhancing axonal survival and regeneration.

Embodiment 20: A method for treating spinal cord injuries by preventing secondary axonal degeneration and promoting functional reconnection.

Embodiment 21: A method for mitigating traumatic brain injury and stroke-related axonal damage through neuroprotective strategies, wherein the neuroprotective strategies comprise white matter preservation.

Embodiment 22: A method for preserving and restoring optic nerve function following traumatic, idiopathic, or disease-induced damage, wherein the disease-induced damage is glaucoma-related degeneration.

Embodiment 23: A method for treating a neurodegenerative disease, wherein the neurodegenerative disease is ALS, Alzheimer's, Parkinson's, MS, idiopathic degeneration, or glaucoma, the method comprising inhibiting axonal degeneration through SARM1 pathway modulation, mitochondrial support, and oxidative stress reduction.

Embodiment 24: A method for treating an autoimmune neuropathy, wherein the autoimmune neuropathy is chronic inflammatory demyelinating polyneuropathy (CIDP) or Guillain-Barre Syndrome, the method comprising stabilizing axonal integrity and reducing inflammatory damage.

Embodiment 25: The method of embodiment 2, wherein small-fiber axon protection is applied to fibromyalgia or chronic fatigue syndrome by stabilizing small-fiber nerve integrity and modulating neurogenic inflammation.

Embodiment 26: The method of embodiment 2, wherein axonal survival mechanisms mitigate post-viral neuropathies, including long COVID-associated small-fiber neuropathy, by enhancing axonal survival and reducing neuroinflammation.

Embodiment 27: A method for preserving musculoskeletal functionality after muscle, bone, tendon, or ligament injury by maintaining neuromuscular and connective tissue integrity and preventing neurogenic degeneration.

Embodiment 28: The method of embodiment 15, wherein the axon-preserving transplantation method enhances organ and limb transplant outcomes by preserving donor axons, nerve grafts, and end targets to sustain transplant viability.

Embodiment 29: A method for mitigating neurogenic degeneration in microgravity environments by preserving axonal integrity and neuromuscular connectivity.

Embodiment 30: A method for preventing muscle wasting and neuromuscular deterioration in conditions such as cachexia, sarcopenia, or prolonged immobilization by sustaining axonal support and trophic factor signaling.

Embodiment 31: A method for treating complex regional pain syndrome by stabilizing peripheral axons and modulating neurogenic inflammation.

Embodiment 32: A method for treating chronic neuropathic pain by enhancing axonal survival, reducing excitotoxicity, and modulating neuroinflammatory pathways to prevent maladaptive pain signaling.

Embodiment 33: The method of embodiment 2, wherein axonal survival mechanisms are applied to chemotherapy-induced neuropathy by enhancing axonal survival, stabilizing mitochondrial function, and reducing oxidative stress.

Embodiment 34: The method of embodiment 2, wherein axonal survival mechanisms are applied to radiation-induced neuropathy by preventing axonal degeneration and preserving neuromuscular connectivity.

Embodiment 35: A method for treating peripheral nerve compression syndromes by preserving axonal structure, reducing ischemic stress, and enhancing nerve regeneration.

Embodiment 36: A method for treating diabetic neuropathy by stabilizing mitochondrial function, reducing oxidative stress, and preventing axonal degeneration.

Embodiment 37: A method for treating metabolic neuropathies by sustaining mitochondrial function, reducing oxidative stress, and preserving axonal integrity.

Embodiment 38: The method of embodiment 6, wherein retrograde axonal transport mechanisms are applied to neurogenic bladder dysfunction to preserve autonomic and somatic innervation.

Embodiment 39: The method of embodiment 7, wherein trans-synaptic axon protection is applied to neurogenic cardiovascular dysfunction by preserving autonomic nervous system integrity and stabilizing baroreceptor reflex pathways.

Embodiment 40: A method for mitigating post-stroke motor dysfunction by preserving axonal pathways, enhancing synaptic plasticity, and supporting neuromuscular rehabilitation.

Embodiment 41: A method for treating pediatric neurodevelopmental and neuromuscular disorders by stabilizing axonal function, promoting early neurodevelopmental connectivity, and preventing progressive axonal degeneration.

### EXAMPLES

### Example 1

Described herein is a review of the physiology, pathophysiology, and clinical challenges associated with peripheral nerve repair, with a focus on the endogenous capacity for regeneration, limitations of this capacity, and the ability of current and emerging repair strategies to address these issues. Notably, this example focuses on mammalian PNI repair, where regenerative capacity is limited compared to certain non-mammalian vertebrates (e.g., amphibians) and invertebrates (e.g., annelids and cephalopods), which often exhibit more robust axonal regrowth mechanisms. Focus is given to a series of next-generation tissue engineering strategies, each designed to overcome specific challenges currently facing the field. This example is also intended to serve as a blueprint for bioengineers and surgeons to aid in the development of new technologies that will enable a multifaceted approach to address the key limitations diminishing the potential for functional recovery after major PNI.

While conventional perspectives have predominantly attributed suboptimal functional recovery following severe peripheral nerve injury to the limited rate of axonal regeneration, emerging evidence demonstrates a more nuanced and multifaceted pathophysiological landscape. This analysis synthesizes current understanding of additional interrelated determinants that influence regenerative outcomes, establishing a proposed framework of challenges that can be addressed for successful therapeutic intervention (Table 1). The translation of regenerative medicine strategies necessitates a comprehensive mechanistic understanding of these interconnected pathophysiological processes, as they collectively determine the efficacy of peripheral nerve repair interventions.

TABLE 1
Challenges Hindering Recovery Following PNI

| *Challenge | Mechanistic | Research | Therapeutic | Opportunities for |
|---|---|---|---|---|
| Priority | Component | Attention | Development | Innovation |
| 1. End-Organ | Maintenance of | Low to | Minimal | Development of |
| Receptiveness | neuromuscular | Moderate | Electrical | targeted end-organ |
| | junction | | stimulation | preservation |
| | architecture | | protocols | strategies |
| | Prevention of | | Exercise | Novel approaches |
| | muscle fiber | | regimens | for maintaining |
| | atrophy | | Basic trophic | tissue architecture |
| | Preservation of | | support | during denervation |
| | sensory end- | | | Integration of tissue |
| | organ viability | | | engineering |
| | | | | principles |
| 2. Schwann Cell | Pro- | Moderate | Moderate | Advanced |
| Environment | regenerative | | Cell therapy | biomaterial designs |
| | phenotype | | approaches | Temporal control of |
| | maintenance | | Biomaterial | cellular |
| | Neurotrophic | | scaffolds | programming |
| | factor | | Growth factor | Enhanced delivery |
| | production | | delivery systems | systems |
| | ECM | | | |
| | organization | | | |
| 3. Neuronal | Retrograde | Low | Moderate | Novel drug delivery |
| Somatic Health | stress response | | Neuroprotective | strategies |
| | Metabolic | | agents | Targeted molecular |
| | support | | Metabolic | interventions |
| | Regenerative | | modulators | Combinatorial |
| | capacity | | Anti- | therapeutic |
| | | | inflammatory | approaches |
| | | | approaches | |
| 4. Axonal | Growth cone | High | Multiple | Enhanced guidance |
| Regeneration | dynamics | | Physical guidance | systems |
| Rate | Cytoskeletal | | strategies | Metabolic |
| | assembly | | Electrical | optimization |
| | Metabolic | | stimulation | Synergistic |
| | support | | Growth factor | therapeutic |
| | | | gradient therapies | combinations |

*Note:
Challenges ranked by magnitude of impact on functional recovery outcomes, with consideration given to current therapeutic limitations and opportunities for innovative intervention.

PNI Repair Across Species: A Comparative Perspective. Unlike mammals, where severed axons undergo rapid Wallerian degeneration and exhibit slow, often incomplete regeneration, many invertebrates demonstrate remarkable anti-degenerative and pro-regenerative capacity. In some invertebrate species, severed axons can survive for months to years without degeneration, allowing for rapid restoration of lost behaviors within days. Following transection, both proximal and distal ends separate by 1-3 mm and seal off quickly, preventing cytoplasmic loss and enabling both segments to remain viable. Delayed degeneration, in some cases up to 7 months after disconnection, is supported in part by trophic factors from adjacent non-myelinating glial cells.

Invertebrate axons also exhibit rapid and directed regrowth. Proximal segments extend at a rate of 1-3 mm/day, selectively reestablishing functional connections with their distal counterparts, which can persist for extended periods. Recovery may occur via direct axonal fusion, formation of gap junctions, chemical synapse formation, or ephaptic interactions. If fusion does not occur, the proximal axon continues regenerating at 1-2 mm/day, guided by signals from the distal segment until it reinnervates its original target, at which point the distal axon typically degenerates.

While mammalian nerve regeneration is often inefficient, the remarkable regenerative strategies observed in invertebrates present valuable insights that can be applied to engineering approaches. The ability to preserve axons long-term, facilitate targeted reconnection, and bypass slow axonal outgrowth highlights strategies for bioengineered solutions in nerve repair. Understanding and harnessing these mechanisms can lead to breakthroughs in mammalian PNI treatments, offering new paradigms or methods for preserving connectivity and restoring function more efficiently.

Responses to Mammalian Peripheral Nerve Injury. To appreciate the complex phenomena of functional recovery, one can have a comprehensive understanding of the PNI response, including regenerative capacity of the proximal neurons, potential for axonal extension across a lesion zone and within the distal nerve sheath, capability of the Schwann cells to sustain the distal pro-regenerative environment, and receptiveness of the muscle for targeted reinnervation. Clinical challenges associated with peripheral nerve repair are described herein with respect to the mechanisms affecting the likelihood of functional recovery following severe nerve injury.

Proximal Neuron Responses to Acute and Prolonged Axotomy. After PNI, neuron cell bodies undergo a series of morphological and molecular changes collectively described as chromatolysis, resulting in the relocation of the nucleus to the cell periphery and dissolution of Nissl bodies in the rough endoplasmic reticulum. These changes are accompanied by altered expression of hundreds of regeneration-associated genes (RAGs), proteins, and transcription factors that are correlated to the regenerative capacity of the proximal neuron after injury, including brain-derived neurotrophic factor (BDNF) and its associated trkB receptor, glial-derived neurotrophic factor (GDNF) and corresponding GFRa1 and RET receptors, and cytoskeleton proteins that are transported to the growth cone for axon outgrowth, such as actin and tubulin.

Proximal neurons need sustained neurotrophic support to maintain a regenerative phenotype and facilitate axon outgrowth through the denervated distal segment and reinnervation of the end target. Without adequate neurotrophic and/or structural support, the regenerative capacity of proximal neurons can diminish, leading to reduced axonal growth, potential neuronal loss, and decreased spinal plasticity, which collectively may lower the ceiling for functional recovery. Endogenous sources of neurotrophic factors are found in the injured nerve proximal to the injury, the denervated distal nerve, and the denervated muscle target. Regenerating axons uptake neurotrophic factors secreted by Schwann cells and other support cells that are then transported to the neuronal cell body in the ventral spinal cord or dorsal root ganglia via retrograde transport.

The extent that axotomy impacts regenerative capacity is dependent on several clinical factors, such as age, pre-existing conditions (e.g., diabetes), modality (e.g., sensory versus motor neuron) and location (e.g., proximal versus distal; cranial versus peripheral). At the time of injury, a large inward flux of extracellular calcium occurs across the ruptured axonal membrane and triggers several biological processes for regeneration. The proximal calcium wave leads to some damaged neurons dying and others surviving but with limited regenerative capacity. Neuron survival is largely influenced by location and neuron subtype. Indeed, sensory neurons are more resilient than motor neurons, and proximal nerve injuries are often more severe than distal nerve injuries. Additionally, delayed surgical repair results in diminished regenerative capacity likely from the lack of distal neurotrophic support. Although delayed repair results in fewer motor axons reinnervating the end target, a subpopulation of motor neurons retain their ability to regenerate following prolonged axotomy. For example, in a clinical case report the authors claimed that after 22 years, axotomized motor neurons were able to extend axons and reinnervate a newly-denervated muscle after repair.

Axonal Response to injury. As noted, the large calcium influx is also partially responsible for neuronal death, following PNI due to increased somal calcium concentration and leading to calcium-induced apoptosis. Immediately after injury, calcium-induced vesicle release seals the injured axonal plasma membrane. Axons disconnected from the proximal cell body transport proteins and transmit action potentials for several hours to days after axotomy. Eventually, the axons and myelin in the distal nerve undergo Wallerian degeneration. During the acute injury phase, the Schwann cells forming myelin sheaths around the axons dedifferentiate and proliferate, and aid in phagocytosis of the axon and myelin debris. As the degeneration process continues, macrophages are the main cells for phagocytosis of the cellular debris. Within hours after injury, axons in the proximal stump demyelinate and degenerate up to the first node of Ranvier, allowing for early growth cone extension along a suitable substrate.

Axons maintain an intrinsic ability to regenerate across an appropriate scaffold, bridging the proximal nerve stump to the distal nerve stump. Moreover, as the proximal neuron undergoes morphological and genetic changes, axons extending from the proximal stump undergo similar changes, depending on their current state (e.g., regenerating, maturing, or pruning). During the regenerative process, axons within the growth cone increase expression of proteins and genes linked to development, such as growth-associated protein (GAP-43), tubulin, and actin. Such changes allow for regenerating axons to sprout several smaller neurites for axonal pathfinding; yet this energetically demanding process, requires substantial support from the proximal neuron and haptic/trophic guidance from distal structures. Over time, axons that prosperously penetrate the distal nerve segment undergo maturation, further myelination, and increased fiber diameter, whereas pruning of smaller axons that did not reach the correct end target occurs.

Distal Schwann Cell Responses to Acute and Prolonged Denervation. Schwann cells provide neurotrophic support to sustain the pro-regenerative state of the proximal neurons and enable growth cone extension and axonal outgrowth. After nerve injury, the loss of axonal contact leads to dedifferentiation of the Schwann cells and may result in swift proliferation proximal to the damaged region and throughout the denervated distal nerve. The dedifferentiation of myelinating and non-myelinating Schwann cells results in the formation of a pro-regenerative environment after the upregulation of several RAGs.

Denervated Schwann cells undergo another morphological change, and form aligned columnar structures on the basal lamina of the denervated endoneurium, described as bands of Büngner, that provide support for regenerating axons through the denervated endoneurium. The pro-regenerative Schwann cells are the major source of neurotrophic factors (e.g., brain-derived neurotrophic factor (BDNF), glial-derived neurotrophic factor (GDNF), neurotrophin-3 (NT-3), and nerve growth factor (NGF)) diffusing proximally towards the growth cone of the regenerating axons to sustain the neuronal somata and drive reinnervation of the distal muscle. Over time, a prolonged period without axonal contact results in the degradation of the pro-regenerative environment; this creates diminished structural support and neurotrophic factors for axonal pathfinding and long-distance regeneration.

Two main factors linked to the deterioration of the pro-regenerative environment after chronic denervation are likely the reduction in the number of Schwann cells and loss of the regenerative phenotype. However, restoring early axonal contact to the distal nerve appears to “babysit” the pro-regenerative environment and enable axonal regeneration and reinnervation.

Muscle Responses to Acute and Prolonged Denervation. After a PNI, the denervated muscle undergoes a series of morphological changes that may limit functional regeneration. In the early stages of denervation, muscle fibers remain viable, but motor units are reduced in the denervated muscle. These changes are accompanied by altered expression of several neurotrophic factors, including but not limited to GDNF, BDNF, NGF, NT-3/4/5, and CNTF.

For a brief period after injury, the denervated muscle can evoke an electrophysiological response depending on the length of the denervated distal segment, suggesting that a neuronally-derived soluble molecule or protein transported to the muscle via axons may preserve the electrophysiological activity of the denervated muscle. A recent study suggests that acetylcholine may prevent morphological changes associated with muscle denervation and increase synaptic integration after injury. Several neurotrophic factors are useful for successful regeneration; however, the BDNF and the trkB signaling pathways are for terminal Schwann cell survival, motor axon sprouting, and neuromuscular junction (NMJ) stabilization and/or pruning. After reinnervation, upregulated BDNF expression and mRNA levels in denervated muscle return to baseline, indicating a role in targeted axonal reinnervation during the regenerative process.

Prolonged denervation leads to muscle atrophy, with myofibril reabsorption, myocyte shrinkage, and collagen expansion in the extracellular matrix (ECM). Denervated muscles become less receptive to functional recovery, likely due to structural alterations that impact muscle contraction, as well as changes in the expression of intramuscular neurotrophic factors for reinnervation. Terminal Schwann cells found within the denervated muscle have a role in muscle reinnervation by guiding regenerating axons and synaptic pruning, and remodeling at the NMJ. After injury, denervated terminal Schwann cells extend to adjacent innervated NMJs, which promote intramuscular axonal sprouting and polysynaptic innervation. Longer periods of denervation are correlated with poor functional recovery and elevated polyneuronal innervation; this is thought to be due to reinnervation of a neuromuscular junction by an “escaped” axon, growing along the terminal Schwann cell processes. Additionally, prolonged denervation results in the detachment of some terminal Schwann cells, which diminishes the likelihood of reinnervation of these abandoned acetylcholine receptors. Therefore, successful functional recovery largely depends on the receptiveness of the denervated muscle and presence of pro-regenerative Schwann cells.

Clinical Factors Affecting Regeneration and Recovery. Several clinical factors have been identified that influence regeneration and functional recovery, such as age and patient health, as well as type of injury (e.g., neuropraxia, axonotmesis, neurotmesis), location of injury (e.g., proximal or distal nerve), repair time (e.g., acute or delayed repair), and defect length in segmental nerve damage (e.g., short or long gap nerve). In this section, regenerative capacity of the proximal neuron, distal nerve segment, and muscle will be reviewed based on these clinical factors.

Age and Health of the Patient. A factor in predicting positive recovery is patient age. Notable sensorimotor functional recovery is well-documented in pediatric cases following median and/or ulnar nerve transection and repair. While the exact mechanism in these cases remains unclear, children have a superior capacity for functional recovery compared to adults that is likely related to shorter regenerative distances to the muscle and/or sensory end targets, higher intrinsic capacity for axonal extension, and greater spinal cord plasticity. Underlying patient health is a contributing factor to functional recovery after an injury, which may be at greater risk for poor outcomes after PNI, for instance, patients with diabetes or additional polytrauma.

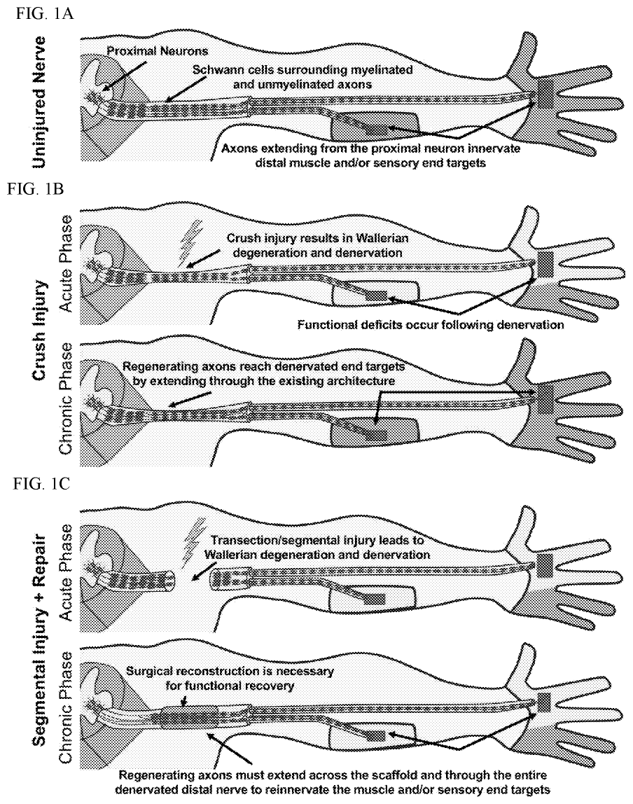

Severity of Nerve Injury. The severity of nerve injury is primarily dependent on the extent of damage to the nerve's architecture (FIG. 1). There are three levels of injury severity according to the Seddon classification: neurapraxia, axonotmesis, and neurotmesis. Neurapraxia is a transient disruption of ion flow due to local myelin damage or ischemia but without axonal injury, and spontaneously resolves after remyelination. Axonotmesis typically results from a crush or stretch injury in which axons are damaged without disrupting the nerve's connective tissue. In these cases, after Wallerian degeneration, the axons regenerate through intact nerve tissue to the end-target. Neurotmesis is the most severe injury and includes complete transection of axons and surrounding connective tissue. Surgical intervention is useful in cases of neurotmesis, and a scaffold may be needed to bridge the nerve stumps and enable regeneration. While the Seddon classification system provides mechanistic insight, Sunderland established an injury grading scale that offers greater specificity for recovery prognosis. Sunderland Type I correspond to Seddon's neurapraxia, Type II-IV correspond to axonotmesis, and Type V corresponds to neurotmesis. Although Sunderland grading scale provides additional prognostication value, clinical scenarios rarely result in one injury type. To address this inconsistency, Mackinnon and Dellon added a Type VI injury corresponding to a mixed injury, which is a common clinical presentation. Of note, Sunderland Type IV-VI, and sometimes Type III, do not resolve spontaneously and require surgical intervention to reconnect the proximal and distal stumps.

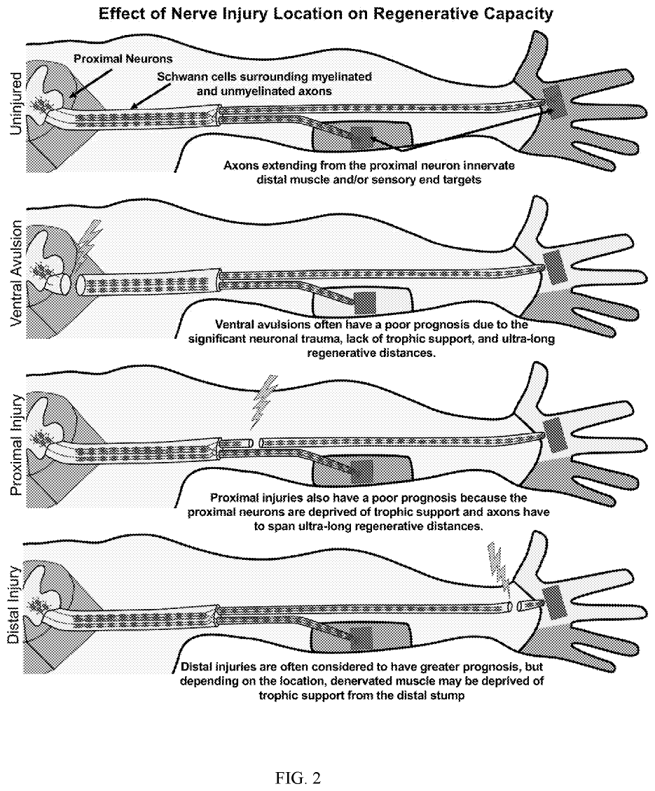

Location of Nerve Injury. Proximal nerve injuries, such as those to the brachial plexus or large caliber peri-midline nerves, often result in poor functional recovery following surgery due to the slow rate of axon regeneration (1 mm/day or 1 inch/month) and long distance (600-1200 mm or 2-4 feet) to the end target (FIG. 2). Distal nerve injuries, like those to the median or ulnar nerves at the wrist, have a shorter regenerative distance of around 100 mm and typically result in better recovery, with axons able to reinnervate the hand muscles within 100 days. However, proximal nerve injuries, like brachial plexus injuries, may require axons to span regenerative distances greater than 1 meter (1000 mm) to reach the hand, which would require 2-3 years. However, as described herein, the prolonged period without proximal axons innervating the end target(s) and lack of axonal connection with the denervated structures diminishes the likelihood of meaningful functional recovery. As such, axonal regeneration and distal target (i.e., muscle) health are not inherently sustained over the 2-3 year period useful to achieve functional regeneration following brachial plexus injury and surgical repair, resulting in poor and incomplete recovery. Shorter regenerative distances to the denervated target and the related preservation of proximal neurons are thus components that underlie better functional recovery after nerve injuries in children. In general, repair of distal nerve injuries results in better recovery due to the shorter regenerative distance, even in cases of prolonged denervation.

Length of Segmental Nerve Defect. In cases of segmental nerve injury, functional recovery is inversely related to the length of the graft (i.e., shorter gap repairs have a better prognosis than long gap nerve repair). In adult humans, the gap length is 4-5 cm, which is the maximum distance that an acellular graft can support nerve regeneration and reinnervation. The likelihood of graft failure increases as the graft distance approaches or exceeds this gap length due to several interrelated factors, including poor axon regeneration, increased Schwann cell senescence, incomplete ECM deposition, poor revascularization, inadequate macrophage infiltration, and diminished axon maturation and myelination. Long gap nerve injury greater than the gap often results in poor functional recovery due to prolonged denervation of the distal nerve, contributing to the diminished pro-regenerative capacity of the distal Schwann cells and decreased muscle receptiveness described herein. Notably, acellular scaffolds result in slower axonal regenerative rates compared to autografts, resulting in greater periods of denervation and diminished functional recovery (also see sections on “Nerve reconstruction strategy” and “Current clinical approaches for peripheral nerve repair” below).

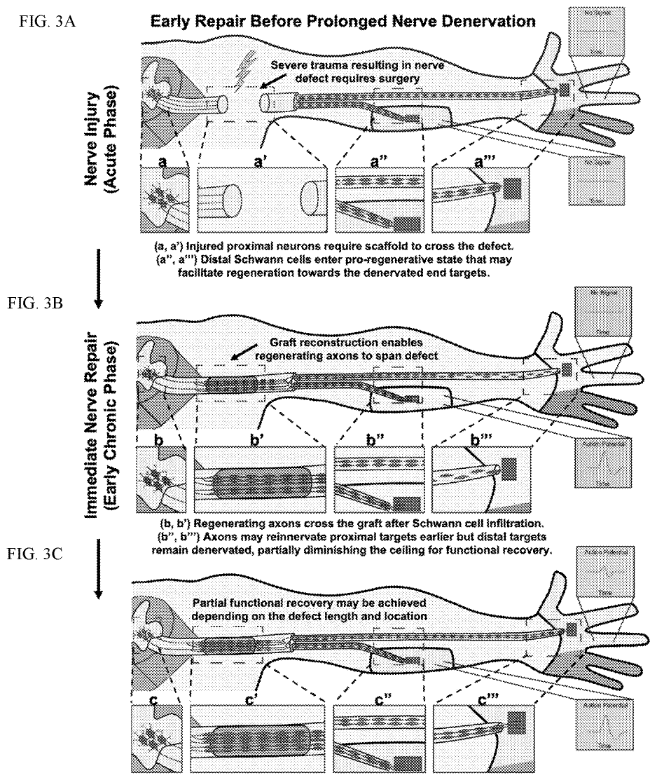

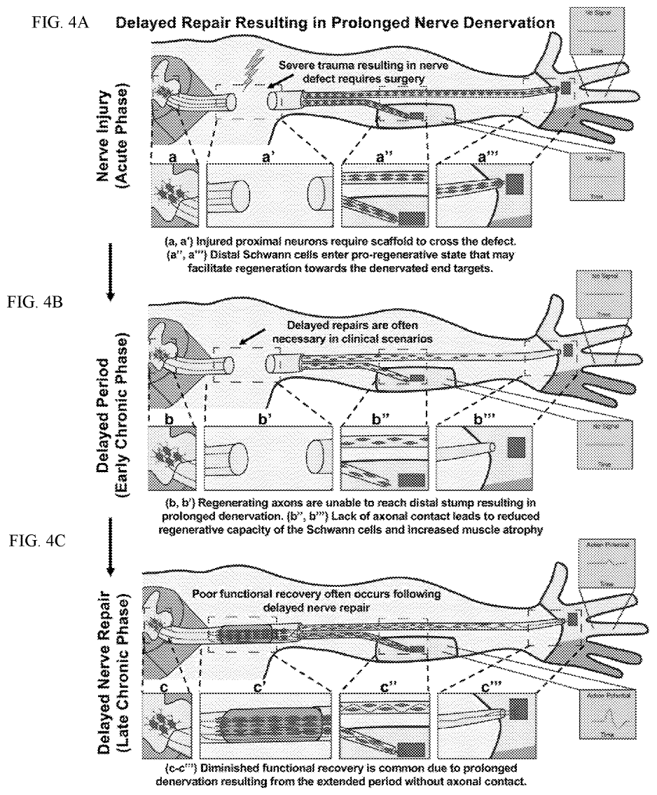

Time to Nerve Repair. Early surgical intervention is associated with greater motor and sensory functional recovery compared to delayed intervention (FIGS. 3 and 4). There are several factors that contribute to poor recovery after delayed nerve repair, including a decrease in the regenerative capacity of proximal neurons, loss of spinal cord plasticity, and reduced receptiveness for reinnervation at the end target. Proximal neurons can maintain a regenerative phenotype for a period after injury; however, the regenerative capacity decreases over time without retrograde neurotrophic support and lack of contact with a suitable end target. Additionally, delayed repair results in prolonged periods without axonal contact with the denervated nerve and muscle that can also lead to the loss of the pro-regenerative environment secreted by Schwann cells and decreased potential for muscle reinnervation, reducing the likelihood of functional recovery (FIG. 4).

Nerve Reconstruction Strategy. For severe nerve injuries that result in a transection or segmental defect (Sunderland Type V), a tensionless direct nerve repair is preferred for gaps under 1 cm, while nerve grafting may be useful for larger gaps. Surgical interventions aim to restore functionality to the affected area by bridging disconnected nerve stumps with a suitable graft that serves as a scaffold for regenerating axons. Nerve autografts are the gold standard for gaps 1-10 cm, while vascularized nerve grafts may be used for larger gaps. In some cases, a tensionless direct nerve repair may be possible for short segmental defects by nerve dissection and transposition, such as the ulnar nerve around the elbow, but for longer defects, the chances of functional recovery may be diminished due to prolonged denervation and degradation of the environment conducive to nerve regeneration.

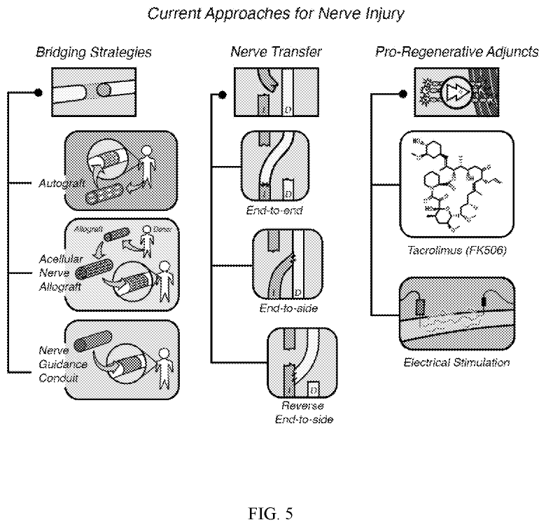

Clinical Approaches for Peripheral Nerve Repair. Clinically available nerve repair strategies and the effects of these approaches on the overall regenerative capacity with respect to the axotomized proximal neuron, denervated Schwann cells, and denervated muscle are described herein. Examples of various peripheral nerve strategies are summarized in FIG. 5.

Autografts: A natural living scaffold for regenerating axons. Autografts are considered the gold standard for nerve repair because they allow axons to interact with a living scaffold that is similar in structure to the denervated nerve. A healthy donor nerve from the patient's own body is used as the scaffold for nerve repair. Autografts are considered living scaffolds because they provide regenerating axons with a bolus of neurotrophic support from endogenous Schwann cells and other cell types as well as structural support from the anisotropic donor nerve architecture. Schwann cells in the donor nerve of the autograft rapidly dedifferentiate and form the pro-regenerative bands of Büngner that enables rapid axon outgrowth across the segmental defect. While autografts are highly effective, they do require an additional surgical site to harvest the donor nerve and there may be limitations on the length available. Donor nerve(s) are selected based on the caliber of the injured nerve and multiple cable grafts (i.e., smaller diameter grafts bundled in parallel) may reduce diameter mismatch. Despite some evidence suggesting motor nerves may provide greater functional recovery when used as donor nerves compared to sensory nerves, an expendable sensory donor nerve is typically used to avoid motor deficits, with the sural nerve in the back of the leg being the most common sensory nerve used for this purpose. Sensory nerve autografts may also protect the denervated muscle with trophic support, increasing the likelihood for successful reinnervation.

Hollow Nerve Guidance Conduits: Protective environment for regenerating axons. Nerve guidance conduits (also referred to as nerve guidance tubes) are clinically available alternatives to autograft repair for bridging less severe segmental defects, such as distal nerve injury and/or defects under the gap length (3 cm defect in humans). After repair, as Schwann cells in the proximal and distal stump dedifferentiate, a fibrin cable starts to form within the graft, promoting vascularization and pro-regenerative macrophage infiltration, with concomitant Schwann cell proliferation and migration into the conduit. After ECM is deposited within the graft zone, Schwann cells form bands of Büngner that allow axonal regeneration into the conduit. Conduits lack a parallel basal lamina, which may result in unaligned bands of Büngner, with reduced effectiveness of axonal regeneration across the graft. Due to the acellular nature of nerve conduits, proximal neurons—the source of locally regenerating axons—are deprived of neurotrophic factors for entering and maintaining a pro-regenerative state. In addition, regenerating axons need a substrate for growth and thus cannot proceed across the gap until Schwann cell infiltration and organization occur, resulting in slower rates of axonal regeneration across an acellular graft (typically 0.10-0.25 mm/day) and thus prolonged denervation of the distal nerve and muscle target.

To date, none of the several commercially available conduits have demonstrated superior levels of axon regeneration and functional recovery compared to the autograft. However, over the last few decades, minimal design criteria for nerve guidance conduits have been established. For instance, effective nerve conduits should be made using porous biomaterials for waste and nutrient diffusion, with an ideal pore size between 5-30 m to minimize excessive fibrosis and inflammatory cell infiltration. This is important as the distance between nerve stumps increases. For instance, while nutrients can reach the center of the graft with short defects via longitudinal diffusion from the terminal ends, regeneration across longer defects requires mass transport across the conduit wall along the length of the graft. The conduit can also provide structural support for infiltrating cells to facilitate axon regeneration during the early regenerative phase, but then degrade and resorb during the chronic regenerative phase to minimize risk for deleterious foreign body response and damage to the underlying/surrounding tissue. The degraded byproducts should be biocompatible, non-cytotoxic, and non-immunogenic. Also, timing of degradation can be tuned appropriately as sufficient regeneration, ECM deposition, and structural support from infiltrating host tissue are useful across the conduit space prior to biomaterial dissolution to avoid graft failure, loss of patency, and/or fibrotic infiltration. Conduits should be flexible and kink-resistant, especially for repairs spanning articulating joints, such as the median nerve coursing around the elbow. Note that these criteria are focused on indirectly supporting axon outgrowth, which, as described herein, needs initial Schwann cell infiltration across the conduit. Unlike living scaffolds such as autografts, nerve guidance conduits do not address proximal neuron health, Schwann cell maintenance, and end-target vitality, limiting their application to less severe indications, specifically short gap repair or distal nerve injury.

Acellular Nerve Allograft: Structural support for regenerating axons that resemble native nerve architecture. Commercially available acellular nerve allografts (ANAs) are a popular alternative to nerve autografts, generated by decellularizing cadaveric nerves to prevent an immunogenic response, while providing host Schwann cells and regenerating axons with a substrate resembling the native nerve architecture. Although endoneurial tubes are present in ANAs, host Schwann cells in the proximal and distal stumps can dedifferentiate and migrate into the graft to enable the environment required for axonal regeneration. Several studies investigating whether ANAs are suitable for long gap nerve repair reported that while Schwann cells initially proliferate and migrate into the graft, they eventually begin to express markers of senescence, such as β-galactosidase and p16^{INK4A} and thus may have an insufficient number of proliferative cycles to re-populate the graft zone. In these cases, there may simply be an insufficient quantity of host Schwann cells to physically bridge the graft, thus failing to provide a continuous substrate for axonal re-growth from the proximal to the distal stumps. While ANAs structurally resemble the autograft, the lack of an endogenous supply of Schwann cells leads to diminished neurotrophic support and slow axonal extension, diminishing the ceiling for functional recovery. Therefore, without modification, ANAs are unable to offer support for injured neurons, pro-regenerative distal Schwann cells, denervated muscle, or sensory end targets. Notably, recent studies have reported poor functionality following ANA reconstruction of large caliber nerves corresponding to the defect length. Notably, ANAs are often utilized by plastic and orthopedic hand surgeons for clinical management of various nerve repairs, including short-gap repairs close to end targets and management of painful neuromas in continuity. For the latter application, following neuroma excision, one end of the ANA is secured to the proximal stump of the transected nerve and the other end is grafted to a nearby muscle that serves as an end target for the regenerating axons that prevents fibrotic tissue formation. However, as described herein, ANAs are contraindicated for long-gap nerve repairs due to the inherent limitation presented by Schwann cell senescence, causing incomplete Schwann cell migration/infiltration into the graft zone, resulting in incomplete axon regeneration across the graft, loss of the pro-regenerative environment distal to the graft, and thus lower levels of functional recovery than would be attained using autograft repairs.

Nerve Transfers: Ectopic axons for early reinnervation of the distal nerve and/or muscle. Although autografts can speed up axon regeneration in short gaps and therefore reduce denervation effects in the distal nerve, in cases like proximal or long gap nerve injuries, axons face longer distances to regenerate, inexorably leading to extended periods where distal targets (e.g., muscles) lack axonal contact. To address these issues, innovative surgical approaches using nerve transfers were developed for complex PNIs. Nerve transfers are used in specific cases in an attempt to achieve greater functional recovery with early reinnervation of the distal nerve segment and muscle end target with rerouted axons from an otherwise uninjured (healthy) nerve. Several variations of nerve transfers are currently used clinically, including end-to-end (ETE), end-to-side (ETS), or supercharged (reverse) end-to-side (SETS). Nerve transfers provide the denervated distal nerve with an exogenous axon source and/or trophic support as an alternative approach to reinnervate the injured muscle and/or the sensory end-target. Axons and neurotrophic factors from the previously uninjured donor nerve enter the denervated nerve, maintaining and/or increasing the regenerative capacity of the distal Schwann cells and muscles. Nerve transfers are often used in high (i.e., closer to midline) proximal nerve injuries that require long regenerative distances or for distal nerve injury sacrificing a redundant nerve to reinnervate the denervated nerve. Unlike ETE and ETS, SETS are considered an adjunctive approach to a primary proximal nerve repair to provide donor axons for early reinnervation of the denervated distal muscle. While nerve transfers are promising, a small subset of cases are found suitable for a nerve transfer procedure and they each require sacrificing a healthy nerve or fascicle(s) to reinnervate an injured nerve, which may cause additional functional deficits and increase risk of painful neuroma formation.

Direct Neurotization of Muscle (DAM): Ectopic source of neurotrophic factors and/or early axon reinnervation. In cases where there is distal nerve stump and/or innervation point in the muscle is damaged yet the gap between the nerve and muscle is small, direct neurotization of muscle (DNM) can be completed by directly suturing a transected nerve to a denervated muscle, without the use of a nerve graft. By attaching the donor nerve to a muscle, axons can form neuromuscular junctions and secrete trophic factors to mitigate detrimental effects associated with prolonged denervation. While DNM has some utility, these procedures may not provide as much functional recovery compared to more conventional direct nerve repairs.

Pro-Regeneration Adjuncts: Extrinsic neuroenhancers for accelerating axonal outgrowth, maturation, and/or reinnervation. Tacrolimus (FK506) and electrical stimulation are two adjunctive strategies that have been shown to improve functional recovery after nerve repair by increasing axonal outgrowth and maturation. FK506 activates the MAPK/ERK pathway by binding to immunophilin receptors found on neurons and increasing the expression of growth-associated proteins, which promotes axonal outgrowth, prevents prolonged denervation, and enables earlier reinnervation. Notably, systemic FK506 administration led to functional recovery after ANA repair; however, it is a potent immunosuppressive agent that can cause severe side effects, which has led to the exploration of alternative pro-regenerative approaches. Electrical stimulation appears to enhance the number of proximal neurons with actively regenerating axons by inducing a rapid calcium influx and activating the MAPK/ERK pathway, along with upregulation of BDNF and its trkB receptor. This leads to increased axonal sprouting and accelerated axonal outgrowth, which can hasten reinnervation of the denervated distal nerve and improve sensory and motor function. While electrical stimulation has shown promise for short gap nerve repair, its effectiveness for long gap nerve repair is less clear. Although stimulation after short gap repair may improve the regenerative capacity of both proximal motor and sensory neurons, electrical stimulation after long gap repair appears to preferentially enhance motor axon regeneration. However, even with electrical stimulation, functional recovery following long gap nerve repair remains challenging, as axons can span vast regenerative distances before detrimental effects of prolonged denervation diminish hope of reinnervation.

Other approaches that have been explored to improve functional recovery after nerve repair include exercise and muscle electrical stimulation. Exercise has been shown to increase intramuscular trophic support and enhance nerve regeneration, but the optimal intensity and duration of exercise for improving functional recovery after nerve repair is not well understood. Muscle electrical stimulation may enhance functional recovery after nerve repair by enabling greater nerve regeneration and earlier muscle reinnervation. Although the exact mechanism remains unclear, increased intramuscular levels of pro-regenerative trophic factors (BDNF and GDNF) have been reported following muscle electrical stimulation after prolonged denervation in a rodent model. Another study proposed that elevated Schwann cell-mediated autophagy following muscle electrical stimulation leads to greater myelin clearance and functional recovery after nerve injury.

PEG-Mediated Axonal Fusion: Immediate restoration of axonal connectivity after acute injury. Polyethylene glycol (PEG) is a fusogen that has gained attention as an adjunctive treatment for peripheral nerve coaptation. Under physiological conditions, the membranes of transected axons undergo calcium-dependent budding followed by Wallerian degeneration. By transecting a nerve in calcium-free hypotonic saline and then applying PEG, transected axonal membranes remain open and can fuse together to immediately restore axonal connectivity. Compound action potentials can be evoked across the injury zone, indicating the restoration of electrophysiological activity. PEG fusion has been shown to improve axon and muscle morphometry, functional response, and behavioral recovery in several studies, although the exact chronic mechanism is not fully understood and it is unclear if sustained axonal fusion is useful after immediate repair. However, a major limitation to the widespread adoption of PEG for peripheral nerve repair is that significant Wallerian degeneration occurs by 3 days after injury, preventing potential fusion to the distal nerve beyond relatively acute time points. Also, it remains unclear if any initially fused axons persist chronically. Clinical trials are ongoing to evaluate the efficacy of PEG-mediated fusion following acute nerve injury. Data from the earliest clinical trial shows modest improvement with two-point discrimination.

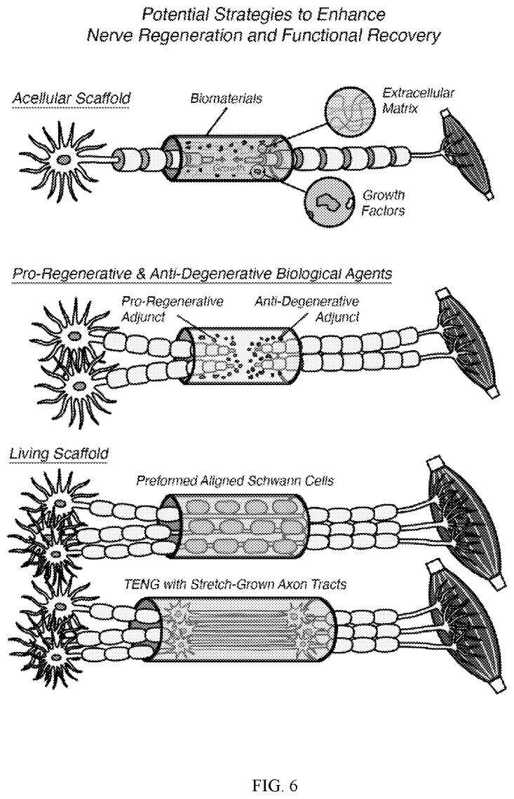

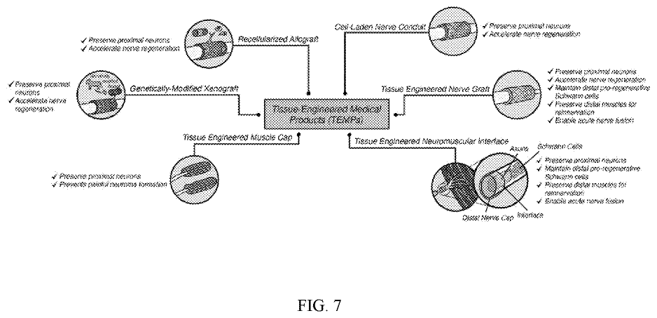

Nerve Reconstruction Without Sacrificing Healthy Nerves. New nerve reconstruction paradigms should strive for the near-term goal of matching levels of functional recovery achieved by gold standard approaches (e.g., autografts for bridging segmental defects and/or nerve transfers to babysit distal structures) without the need to deliberately create additional deficits through the act of acquiring donor nerve/fascicles. In turn, next-generation nerve reconstruction paradigms should have the long-term goal of exceeding levels of functional recovery attainable with current strategies, ideally enabling full functional recovery irrespective of nerve injury severity, location, or length. Achieving these long-term goals will involve a range of therapeutic approaches used in combination and will likely center around the application of tissue-engineered medical products (TEMPs). TEMPs are three-dimensional functional tissue-like grafts comprised of living cells (e.g., neurons/axons, Schwann cells) and/or bioactive factors (e.g., neurotrophic factors, cell-adhesion molecules)—generally with an anisotropic architecture—embedded within a biomaterial scaffold or decellularized tissue. These products are designed to interact with the body and enhance its intrinsic regenerative potential, serving as an alternative to autografts for nerve repair or nerve transfers for distal babysitting. Emerging TEMP technologies are being developed to improve functional recovery after PNI by increasing and/or sustaining the regenerative capacity of the body. FIG. 6 and FIG. 7 summarize TEMP technologies that employ advanced biomanufacturing or cell therapies and their use in nerve repair.

Preserving the regenerative capacity of the proximal neurons during chronic axotomy. One approach to improve functional recovery following PNI is using trophic factors to promote neuronal survival and associated regenerative outgrowth. Examples of trophic factors shown to be effective in promoting sensory neuron survival are NGF and NT4/5, while GDNF, BDNF, CNTF, NT-3, and FGF-2 are effective in promoting motor neuron survival. Growth factor replacement strategies may also help to promote neuronal survival and facilitate spinal plasticity, but the trophic factor(s) and delivery method for improving functional recovery are still unknown. In one study, a GDNF-soaked sponge applied to the proximal nerve stump promoted neuronal regeneration. However, the lack of temporal control is a major limitation. For instance, while prolonged GDNF delivery may improve survival of the projecting neurons, it may also result in the “candy store” phenomenon, whereby axons prefer to stay in the graft region rather than extend to the distal nerve. Therefore, TEMP development should consider adjunctive growth therapies with temporal control.

Another approach is the use of regenerative peripheral nerve interfaces (RPNIs) to improve the regenerative capacity of proximal neurons for delayed repair by providing a temporary physiological end target. When a nerve is injured, proximal neurons enter a pro-regenerative state and spontaneously sprout axons from the proximal stump in search of a physiological end target, such as muscle or sensory end organs. However, this process can result in the formation of a painful neuroma, made up of dense fibrotic tissue and disorganized hypersensitive axons, Schwann cells, and other cells. RPNIs are created by implanting the proximal nerve stump into free, devascularized muscle tissue, which serves as surrogate end target for sensory and motor axons. While this approach has been used extensively for neuroma prevention and as a peripheral nerve electronic interface, it is not clear if RPNIs preserve the regenerative capacity for delayed nerve repair. The development of engineered muscle tissue to simultaneously prevent neuroma formation and maximize regenerative capacity prior to delayed nerve repair is contemplated. Additionally, engineered muscle tissue may be further augmented with electrical stimulation techniques.

In addition to trophic factors and muscle grafts, TEMPs comprised of exogenous dorsal root ganglia explants have been shown to preserve spinal motor neuron health at two weeks after sciatic nerve repair. In this study, the rat sciatic nerve was repaired using stretch-grown dorsal root ganglia neuronal constructs with highly aligned axonal tracts (referred to as tissue engineered nerve grafts or TENGs), unorganized dorsal root ganglia neurons, the gold standard autograft, and conduit (acellular) controls. Remarkably, the use of TENGs or autografts resulted in the survival of the full complement of spinal motor neurons (i.e., motor neuron counts matched uninjured control animals), whereas a modest reduction in motor neurons was observed in animals receiving unorganized neuron constructs and a dramatic reduction (60%) in motor neurons was seen in animals repaired with standard nerve guidance conduits. These results suggest that factors associated with living anisotropic scaffolds (e.g., TENGs and autografts) provide suitable structural and trophic support to improve neuronal health and thus regenerative capacity.

Advanced Nerve Guidance Conduits: Acellular scaffolds for segmental defects. The evolution of acellular nerve guidance platforms represents a progression in regenerative medicine, marked by distinct developmental phases addressing increasingly sophisticated biological requirements. Contemporary bioengineering and material science approaches have transcended basic tubularization to incorporate multiple functionalities: structural guidance, biochemical support, and precisely engineered internal architectures featuring isotropic or anisotropic ECM protein fillers with controlled growth factor delivery systems.

The biomaterial landscape encompasses both natural and synthetic platforms, each offering distinct advantages for clinical translation. Natural biomaterials—including collagen, gelatin, silk, chitosan, and hyaluronic acid—provide inherent bioactivity and cell-adhesion properties, though their implementation is frequently constrained by suboptimal mechanical characteristics and accelerated degradation kinetics. Notable exceptions include silk-based platforms, particularly spider silk derivatives, which have demonstrated enhanced mechanical stability and capacity to support sustained Schwann cell proliferation in both in vitro systems and large-animal models. Synthetic polymers, including poly(glycolic acid), poly(lactic-co-glycolic acid), and innovative tyrosine-derived polycarbonates (TyrPC), offer superior control over degradation profiles and mechanical properties. The development of braided architectures has proven particularly significant, providing kink resistance for applications spanning mobile anatomical regions, with recent studies demonstrating promising regenerative outcomes in both rodent and porcine models.

The efficacy of acellular conduits fundamentally depends on their ability to support coordinated cellular infiltration and matrix deposition. This process uses orchestrated migration of multiple cell types-Schwann cells, macrophages, fibroblasts, and endothelial cells—with successful outcomes heavily influenced by gap length and internal scaffold architecture. While traditional single-lumen designs supplemented with ECM proteins have shown efficacy in short gaps, their limitations become evident in longer defects. Recent innovations in anisotropic internal architectures that more closely mimic native nerve structure have demonstrated improved cellular infiltration patterns, though systematic evaluation across varying gap lengths remains useful.

Technical barriers to clinical translation persist, particularly in long-gap repairs (>4-6 cm), where outcomes consistently fail to match autograft standards due to Schwann cell senescence. Recent advances in sustained delivery systems, exemplified by poly(caprolactone) conduits with double-walled PLGA/PLA microspheres for controlled GDNF release, have demonstrated functional recovery in both rodent (1.5 cm sciatic nerve) and non-human primate (5 cm median nerve) models. However, successful clinical implementation requires addressing several interconnected challenges: optimization of mechanical properties across anatomical sites, preservation of biological functionality during sterilization, and development of scalable manufacturing processes.

Vascular considerations have emerged as a determinant of regenerative success, with the hypoxia-driven secretion of VEGF by infiltrating macrophages initiating vascular channel formation. While direct VEGF supplementation may work, mounting evidence suggests that successful nerve regeneration needs more comprehensive support systems. Thus, integrated approaches addressing multiple aspects of the regenerative environment are needed, including: architectural optimization for gap-length specific applications; enhanced control over cellular infiltration dynamics; improved temporal regulation of growth factor delivery; integration of immunomodulatory functionalities; and development of cost-effective, scalable manufacturing processes. The progression toward clinical implementation demands systematic investigation of these challenges while maintaining focus on practical considerations such as surgical handling characteristics and patient-specific customization requirements.

Tissue Engineered Living Scaffolds: Accelerating axonal regeneration, preserving injured neurons, and/or maintaining the pro-regenerative distal environment. Tissue-engineered living scaffolds designed for nerve repair are generally biofabricated using exogenous cells seeded on or embedded within an anisotropic biomaterial, and thus are capable of secreting neurotrophic factors in response to the microenvironment. Tissue-engineered living scaffolds can be made from a variety of cell types, including neurons and/or Schwann cells—primary or stem-cell derived—macrophages, mesenchymal stem cells (MSCs), or neural progenitor/stem cells.

Autologous cells (i.e., cells from the patient's own body) may be the simplest option for clinical translation, and preclinical and clinical testing of human Schwann cells has shown successful regeneration and functional recovery after long gap nerve repair. While autologous Schwann cells currently have clinical utility, cell isolation requires sacrificing a donor nerve. MSCs may also be an alternative for sourcing Schwann cells at scale, as they can be readily harvested from patients and offer pro-regenerative and immunomodulatory properties for nerve repair. Many MSC caches are available in the body, such as bone marrow, blood, adipose, dental pulp. Notably, gingiva-derived MSCs are a source that can be readily harvested in the clinic and used for many applications. Additionally, gingiva-derived MSCs can be readily differentiated into neural crest cell-like cells and Schwann-like cells and have been used to improve sciatic nerve and facial nerve regeneration in rats. Interestingly, a scaffold-free construct can be fabricated by bio-printing gingiva-derived MSCs and then allowing them to differentiate into Schwann cell-like cells, which then promoted nerve regeneration and functional recovery following facial nerve repair. Neural crest stem-like cells derived from gingiva MSCs also have been reported to improve facial nerve regeneration and functional recovery in rats. Another approach may include using preformed aligned Schwann cells to promote neuronal health and axonal growth. These “tissue engineered bands of Büngner (TE-BoBs)” are fabricated by seeding Schwann cells embedded in collagen in a protective agarose hydrogel, which then rapidly self-assemble into a highly aligned bipolar morphology resembling the native bands of Büngner. Notably, TE-BoBs have been fabricated using gingiva-derived MSCs, further demonstrating their utility as a clinical starting biomass.

Genetic engineering focuses on the ability of cells to overexpress pro-regenerative or immunomodulatory properties. Genetic modification of Schwann cells has been shown to improve axonal growth and functional recovery after nerve injury in animal models. Upregulation of various trophic factors has been attempted; however, successful recovery is associated with precise temporal and spatial cues of many growth factors rather than the overexpression of a single protein. For example, an approach that includes Schwann cell c-Jun overexpression—the master regulator of genetic programming for axonal regeneration—was shown to increase proliferation and migration, and axonal extension. The applicability of these approaches in long gap nerve repair remains unclear. Modifications to cell-based conduits may overcome limitations associated with the “candy store effect” by allowing spatial and temporal control over secretion of neurotrophic factors, which provide regenerating axons with gradient neurotrophic support that also preserves the regenerative capacity of the proximal neuron. Although transplanted cells can regulate trophic secretion by cross-talk with the host microenvironment, genetic modifications for greater temporal control by transplanted cells with “switches” can be extrinsically controlled to overexpress neurotrophic factors.

Stretch-grown tissue engineered nerve grafts (TENGs) are another living scaffold being developed to address major challenges in this field. TENGs are comprised of long axon tracts spanning two neuron populations generated by slowly stretching the populations. TENGs were fabricated by stretch-growing various neuron subpopulations, mimicking the naturally occurring axonal stretch-growth process found during development. Living axons are embedded in a three-dimensional ECM, rolled into tubular form, and inserted within a nerve guidance tube. Nerve regeneration across TENGs uses a different mechanism, termed axon-facilitated axonal regeneration (AFAR) versus the traditional mechanism that uses Schwann cells as a regenerative bridge. AFAR allows regenerating axons to grow along the transplanted TENG axons with accelerated outgrowth and improved functional recovery in rat and pig models of PNI.

While these approaches may replace the autograft, it is unlikely they can lead to superior functional recovery without also resolving the diminished regenerative capacity of the distal nerve. To elevate the ceiling for recovery, it is likely that the challenges associated with peripheral nerve regeneration will need to be addresses. On this front, stretch-grown TENGs may be a strategy to simultaneously accelerate axonal regeneration across the defect while preserving spinal motor neurons. However, unlike autografts, stretch-grown TENGs also provide a source of living exogenous axons that integrate with denervated distal nerve after repair. Here, it was shown that stretch-grown TENGs may be the first nerve repair strategy that simultaneously facilitates axon regeneration across a nerve injury and babysits the pro-regenerative Schwann cells following long gap nerve injury in pigs. While functional recovery was achieved in pigs, further work shows stretch-grown TENG efficacy with a clinically-relevant cell source. To date, stretch-grown TENGs have may be useful as an alternative repair strategy in a clinically-relevant porcine PNI model, featuring large segmental defects (5 cm) and long total regenerative distances to reach distal targets (20 or 27 cm).

Preserving the pro-regenerative state of Schwann cells to mitigate the deleterious effects of prolonged denervation. To date, few studies have examined strategies to preserve the regenerative capacity of the denervated nerve segment. Previously, neurons transplanted in a denervated nerve were shown to preserve the pro-regenerative Schwann cells, prevent muscle atrophy, and promote functional recovery after nerve repair. Ectopic axons in the distal nerve may integrate with denervated Schwann cells and/or muscle to preserve the pro-regenerative environment and improve muscle reinnervation after delayed repair. Several studies show that after preserving this environment with ectopic neurons, greater functional recovery was achieved following a traditional delayed repair by excising the transplanted neurons. After delayed nerve repair, babysitting the denervated nerve enables axonal outgrowth and reinnervation despite significant muscle atrophy; this approach independently preserves Schwann cells rather than just mitigating the detrimental effects of chronic muscle denervation.

To address clinical challenges facing peripheral nerve surgeons, tissue engineered neuromuscular interfaces (TE-NMIs) have also been developed, which are micro-injectable functional engineered tissue containing sensory and/or motor neurons as a strategy to deliver exogenous axons into the denervated distal nerve for delayed nerve repair. TE-NMI axons extended through the denervated nerve and were seen to integrate with denervated Schwann cells for muscle/sensory end target(s). By providing early reinnervation with exogenous axons, TE-NMIs where found to promote the pro-regenerative phenotype of Schwann cells, prevent denervation-induced muscle atrophy, and improve functional recovery following delayed repair. From a translational perspective, TE-NMIs may be a useful adjunct in combination with other primary repair strategies, including autografts, nerve conduits, or TEMPs. Notably, evoked muscle responses were readily elicited following TE-NMI stimulation at 16 weeks post transplantation due to the integration of exogenous axons with the otherwise denervated muscle. As such, rehabilitation electrical stimulation may be a useful adjunctive approach following TE-NMI transplantation- or other babysitting strategies—to promote graft neuronal survival and muscle functionality.

Preserving the receptiveness for reinnervation and effective functional recovery. Prolonged muscle denervation occurs in clinical cases requiring delayed repair or ultra-long regenerative distances. Strategies to prevent prolonged denervation will likely include anti-degenerative small molecules and transplantation of exogenous neuronal tissue (described herein). Recent studies have suggested that the inhibition of connexin hemichannel formation in denervated muscle using small molecules (e.g., boldine) may prevent muscle atrophy and improve reinnervation. Other anti-degenerative therapeutic targets include sterile alpha and TIR motif containing 1 (SARM-1) and mitochondrial transcription factor A (TFAM). SARM-1 is an axonal enzyme primarily responsible for the energy-depletion induced axonal fragmentation component of Wallerian degeneration. Recent studies have shown that SARM-1 inhibition slows Wallerian degeneration, suggesting it may be a useful therapeutic focus for end target babysitting. Another molecular target is Schwann cell-derived TFAM, which has been previously shown to be useful for axon survival after injury. Although anti-degenerative small molecules are promising, meaningful restoration will likely require the use of replacement cells and/or engineered tissue. For example, as noted herein, neuron transplantation in denervated muscle has previously been shown to prevent muscle atrophy and innervate the denervated muscle after nerve injury. Transplanted motor neurons in mice appear to upregulate several pro-regenerative trophic factors with greater functional recovery after delayed nerve repair; however, transplantation at 3 days post-injury did not rescue the denervated distal muscle compared to acutely transplanted controls. Additionally, exogenous axons extending from tissue engineered neuromuscular interfaces (TE-NMI) integrated with denervated muscle, which maintained the evoked muscle response, minimized atrophy, and ultimately allowed greater reinnervation following delayed nerve repair. These data suggest that more effective microtissue-based strategies may improve functional recovery in clinically-relevant scenarios. Indeed, TE-NMI-mediated rehabilitation using non-invasive modalities, such as electrical, optogenetic, piezoelectric, and/or magnetoelectric stimulation may enhance functional recovery.

Surgical Management of Segmental Nerve Repair to Maximize Recovery. The current limitations in peripheral nerve repair stem from the slow rate of axonal regeneration, the progressive degeneration of denervated distal targets, and the failure to maintain a pro-regenerative environment throughout the recovery period. Despite advancements in nerve grafting and biomaterial scaffolds, functional outcomes remain suboptimal, particularly for proximal nerve injuries and delayed repairs.

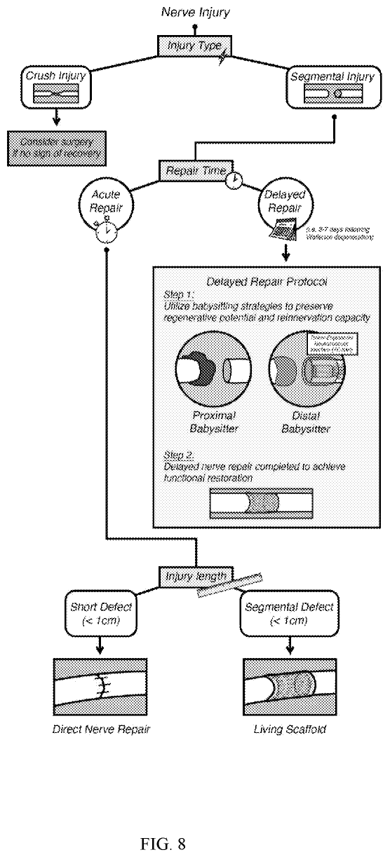

To address these challenges, a next-generation surgical paradigm for nerve repair that integrates biomaterials, cellular therapies, and tissue-engineered medical products (TEMPs) can be used. This strategy incorporates early-stage interventions to preserve neuronal viability and Schwann cell regenerative potential while leveraging advanced grafts to optimize regeneration across segmental defects (FIG. 8).

Acute Nerve Repair: Direct Repair, Grafts, and Tissue-Engineered Enhancements. For acute nerve injuries (e.g., iatrogenic trauma, tumor resection), a tensionless direct repair would remain the gold standard for defects less than 1 cm, as primary neurorrhaphy offers the highest likelihood of functional recovery. However, defects greater than 1 cm require bridging strategies, which may include: autografts, which remain the clinical gold standard despite donor site morbidity and limited availability; engineered nerve grafts, designed to provide a pro-regenerative environment that mimics native nerve structure and biochemical signaling; and TEMPs, used as dynamic bridging grafts to: promote proximal regenerative capacity, ensuring that regenerating neurons remain viable; accelerate axonal regrowth across a segmental defect through bioactive guidance cues; and prevent denervation-induced muscle atrophy and Schwann cell loss, useful for sustaining end-target viability.

In cases of proximal nerve injury, where regeneration may take months to years, a single repair may be insufficient to maintain the integrity of distal targets. Tissue-engineered neuromuscular interfaces (TE-NMIs) represent an adjunctive strategy. By delivering exogenous neurons within the distal nerve sheath, TE-NMIs can provide early reinnervation, functioning as a bioengineered alternative to supercharged end-to-side (SETS) nerve transfers. This temporarily preserves neuromuscular function while awaiting complete nerve regeneration.

Delayed Nerve Repair: A Two-Step Paradigm to Prevent Degeneration. For delayed nerve repair, a two-step surgical approach designed to preserve neuronal viability and Schwann cell function can be used until definitive reconstruction can occur. Traditional delayed repairs suffer from proximal axon dieback, neuroma formation, Schwann cell atrophy, and muscle fibrosis—each of which compromise functional recovery. A two-stage approach as described herein can mitigate these effects by introducing early intervention strategies that stabilize the injury environment, followed by definitive nerve reconstruction when optimal conditions are achieved.
Step 1: Early Intervention with “Babysitting Constructs”. In trauma settings where definitive nerve repair is not immediately possible, tissue-engineered babysitter grafts can be secured to the proximal and/or distal nerve stumps to sustain regenerative capacity while awaiting reconstruction. These constructs can provide temporary neurotrophic support, reducing axonal degeneration and preserving the regenerative potential of the injured nerve.

For example, proximal babysitting strategies can include one or more of the following: cell-based constructs composed of Schwann cells, neurons, or stretch-grown axons could provide neurotrophic support to sustain neuronal health; and tissue-engineered muscle caps could function as temporary surrogate end-targets, allowing regenerating axons to synapse onto muscle fibers while preserving proximal neuronal viability. These strategies can reduce neuroma formation by providing a structured pathway for axonal sprouting instead of chaotic regrowth. Distal babysitting strategies can include: delivering TE-NMIs intraneurally, end-to-side, or within a nerve cap, providing exogenous axonal input to the denervated distal nerve stump and muscle targets. This approach can sustain the regenerative microenvironment while awaiting definitive nerve reconstruction, preventing Schwann cell apoptosis and protecting neuromuscular junction integrity.

Notably, proximal babysitters can be removed at the time of definitive repair, although they may be attached to an acellular graft that remains in place to facilitate integration with the final repair strategy.
Step 2: Definitive Nerve Repair with Advanced Grafts. After days to weeks of babysitter-assisted support, a nerve repair specialist can conduct definitive reconstruction using either traditional autografts or next-generation engineered bridging grafts, tailored to the specific injury through preclinical optimization and customized in-theater delivery. By maintaining the viability of both proximal and distal nerve stumps, this strategy ensures that even delayed nerve repairs are not limited by irreversible degeneration, maximizing functional recovery.

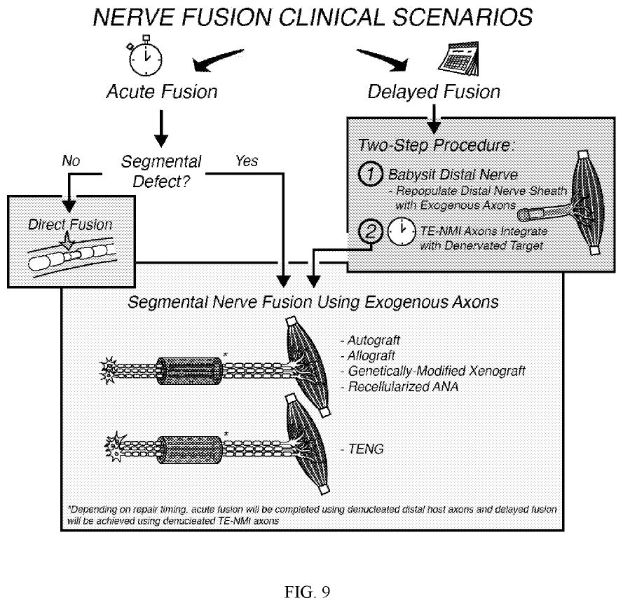

PEG-Fusion and Nerve Reconstruction. The advancement of fusion-based nerve repair is poised to allow widespread clinical adoption of axonal fusion, a potentially paradigm-shifting approach that could restore axonal continuity immediately after injury. In the future, off-the-shelf, axon-based constructs will be readily available for either traditional nerve repair or axonal fusion, depending on the clinical scenario (FIG. 9).

PEG-Fused Allografts: Advancing Nerve Repair Beyond Autografts. Autografts are the gold standard for segmental nerve repair but come with limitations, including donor site morbidity, sensory deficits, and size mismatches that can hinder PEG fusion. While ongoing trials (e.g., NCT04789044) are evaluating PEG fusion with autografts, allografts present a scalable alternative, offering precise size matching and structural support without donor site complications.

Unlike acellular nerve allografts (ANAs; e.g., Axogen's Avance), which are deliberately decellularized and serve as passive scaffolds for regeneration, PEG-fused allografts retain living axonal tracts and intracellular components. These fusion-compatible allografts support immediate axonal reconnection and functional restoration without requiring long-distance regeneration.

Preclinical studies suggest that viable allografts can fuse without immunosuppression. Donor nerve segments transplanted between rat strains have shown rapid functional recovery, indicating that PEG fusion may mitigate immune rejection risks. If allografts integrate successfully without long-term immunosuppression, they could significantly improve accessibility and outcomes in nerve repair.

An important clinical challenge in expanding PEG-fused allografts is preserving axonal viability. Unlike ANAs, which are stable for extended periods at room temperature or frozen, viable allografts require conditions that maintain cell membrane integrity, metabolic activity, and structural organization of axons to ensure they remain functional for fusion. Without proper preservation, axons rapidly degenerate due to ischemia, loss of trophic support, and activation of cell death pathways. Axonal preservation methods that slow metabolic degradation, maintain axonal structure, and extend viability, enabling off-the-shelf, fusion-compatible grafts that can be readily available for clinical use are being developed. Cold storage is useful to overcome the logistical barriers of allograft use in a clinical setting. Currently, fresh allografts can be harvested and transplanted within a short window to retain viability, which is impractical for widespread clinical adoption. By extending the preservation window, cold-stored viable allografts could be pre-prepared, banked, and distributed as needed, eliminating the urgency of immediate transplantation. This would make fusion allografts a practical and scalable alternative, increasing accessibility while maintaining functional efficacy.

Refining preservation techniques, assessing immunogenicity thresholds, and validating long-term outcomes can ensure stable, functional integration. PEG fusion can shift nerve reconstruction from traditional grafting toward a more accessible, scalable, and functionally superior approach.

PEG-Fused Axon-Based TENGs: Bridging Large Segmental Defects. In cases where an autograft is not available or insufficient, PEG fusion using TENGs across segmental defects can be a viable alternative. Unlike standard allografts, TENGs contain pre-aligned axons, offering a living scaffold that supports regenerating host axons (via neurotrophic and structural guidance) and axonal fusion with the host nerve (allowing immediate restoration of electrophysiological function).

Notably, TENGs can be designed to match specific nerve diameters, with axonal densities exceeding those of autografts, increasing the likelihood of successful axonal fusion. Freshly axotomized TENGs may be particularly advantageous, as they can be amenable to PEG-mediated axonal fusion, effectively allowing for functional nerve repair without the need for long-distance axon extension.

Delayed Nerve Fusion: Repopulating Denervated Nerve Segments. While PEG-mediated fusion is currently feasible for acute PNI, its application in delayed PNI remains a challenge. Useful strategies may include utilizing distal babysitting TE-NMIs as an exogenous source of axons to repopulate the distal nerve, permitting axonal fusion beyond acute time points. Notably, noninvasive TE-NMI activation strategies (e.g., electrical, optogenetic, or magnetoelectric stimulation) can be implemented to further enhance axonal integration, prolong the pro-regenerative state of Schwann cells, decrease muscle atrophy, and allow meaningful functional recovery.

Integrating babysitting constructs, tissue-engineered bridging grafts, and axonal fusion techniques, can overcome the constraints of traditional nerve repair, such as slow axonal regeneration, limited donor nerve availability, and incomplete functional recovery.

A Paradigm Shift in Peripheral Nerve Surgery. The combination of PEG-fused allografts, PEG-fused TENGs, and exogenous axon repopulation strategies can lead to delayed nerve fusion, revolutionizing the treatment of segmental and/or chronic nerve injuries. By repopulating otherwise denervated distal nerves, surgeons can achieve functional reconnection in cases previously considered irreparable.

This represents a leap forward in the field of peripheral nerve surgery, shifting the standard of care from traditional nerve grafting to nerve fusion-based techniques. For example, optimizing fusion protocols to ensure consistent clinical success; developing scalable axon-based TENGs to provide reliable, fusion-compatible constructs; and ensuring long-term functional integration of fused axons to confirm durable and lasting repair can improve the standard of care of PNI. With continued progress, PEG-fusion technologies can lead to full functional restoration following nerve injury.

Current surgical interventions for major PNI generally do not result in complete functional recovery, suggesting current strategies promulgate a diminished ceiling for regenerative capacity. Four interconnected physiological responses impact the achievable levels of functional recovery after PNI, including (1) the health and regenerative capacity of the proximal neurons, (2) the extent and rate of axonal regeneration, (3) the capability of Schwann cells to sustain the distal pro-regenerative environment, and (4) the receptiveness of the muscle and/or sensory end organs for appropriate reinnervation. Collectively, these factors diminish the capacity for functional recovery even after state-of-the-art surgical repair, which currently includes reattaching the injured stumps by direct nerve repair (i.e., neurorrhaphy), using a nerve conduit or graft (i.e., autograft), and/or rerouting a nearby nerve (i.e., nerve transfer). It is noteworthy that each of these conventional repair techniques addresses one or two of the aforementioned challenges, and there are currently no comprehensive strategies in clinical use that address each of these challenges. Regrettably, gold standard autograft and/or nerve transfer techniques remain the primary options for surgical repair after severe PNI despite both approaches requiring deliberate transection of an otherwise healthy nerve.

While numerous next-generation strategies have been proposed to improve outcomes following PNI, their successful translation needs an adequate understanding of the clinical challenges and physiological responses impacting functional recovery. Indeed, the utility of emerging repair strategies should be examined through the lens of impacting the aforementioned physiological responses that maximally influence functional recovery. Understanding and addressing these challenges to nerve regeneration is useful for the development and translation of impactful pro-regenerative strategies. The field of peripheral nerve repair is on the precipice of a paradigm shift based on the implementation of TEMPs designed to address simultaneously and comprehensively each of the major physiological responses affecting regeneration and reinnervation, thus providing patients with the chance for full functional recovery where virtually no hope exists with current approaches.

### Example 2

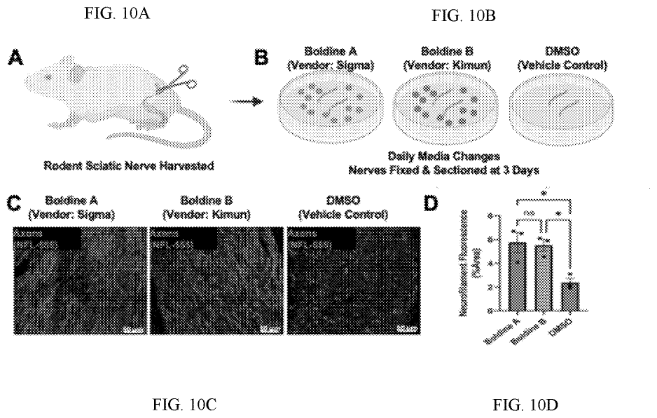

Boldine Preserves Axonal Integrity in an Ex Vivo Sciatic Nerve Degeneration Model. To evaluate whether boldine can prevent axonal degeneration in a biological system, an established ex vivo Wallerian degeneration model using rat sciatic nerve explants was utilized. Following transection, sciatic nerves were maintained in culture with daily media changes and treated with boldine or vehicle control (FIG. 10A). Treatment groups included boldine obtained from two independent commercial sources (Sigma and Kimum) as well as vehicle controls (FIG. 10B).

Axonal integrity was assessed by immunofluorescence labeling of neurofilament light chain (NFL-555), a structural marker of intact axons. At 3 days post-explant, vehicle-treated nerves exhibited extensive axonal fragmentation and loss of organized neurofilament structure, whereas boldine-treated explants displayed greater preservation of axonal architecture with more continuous neurofilament labeling throughout the nerve bundles (FIG. 10C).

Quantification of neurofilament-positive area normalized to total tissue area demonstrated increased axonal preservation in boldine-treated explants relative to vehicle controls (FIG. 10D). Vehicle-treated samples exhibited 2.38±0.37% neurofilament-positive area (mean±SEM; n=3), whereas treatment with Boldine A (Sigma) increased axonal preservation to 5.77±0.84%, and Boldine B (Kimum) to 5.52±0.49% (n=3 per group).

Both boldine preparations produced significantly greater axonal preservation compared with vehicle control (Boldine A vs DMSO, p=0.017; Boldine B vs DMSO, p=0.0237). No significant difference was observed between the two boldine preparations (p=0.9539), indicating consistent biological activity across independent compound sources.

These results demonstrate that boldine treatment significantly slows axonal degeneration in the sciatic nerve explant model.

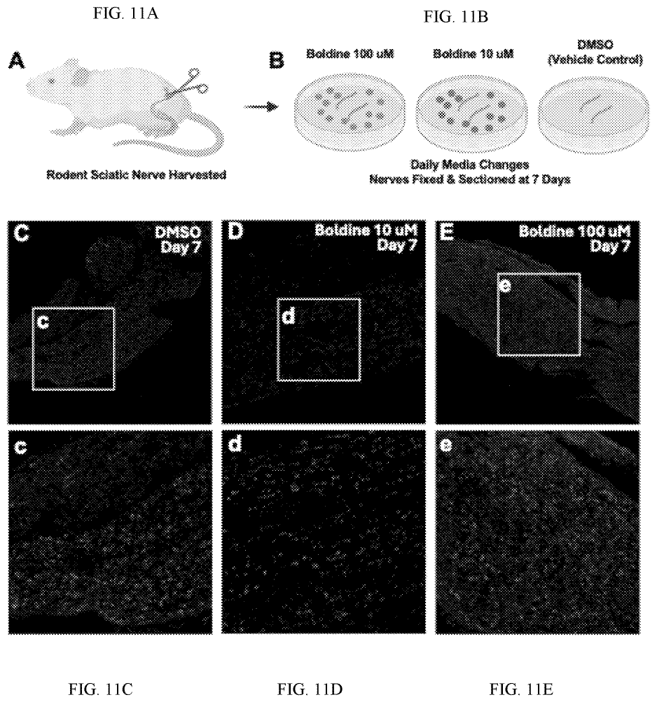

Sustained Axon Preservation Following Boldine Treatment. To determine whether the observed protective effect persisted over longer time periods, sciatic nerve explants were cultured for 7 days in the presence of boldine (10 μM or 100 μM) or vehicle control with daily media replacement (FIG. 11A).

Low-magnification imaging of NFL-555-labeled axons at Day 7 revealed substantial loss of axonal structure in vehicle-treated explants, consistent with progressive degeneration following nerve transection (FIG. 11B). In contrast, explants treated with boldine retained visibly greater axonal organization and neurofilament labeling.

Higher-magnification images confirmed that boldine-treated nerves maintained more continuous axonal structures compared with DMSO-treated controls, which displayed fragmented and punctate neurofilament labeling indicative of axonal breakdown (FIGS. 11C-E).

Together, these findings demonstrate that boldine preserves axonal structure in an ex vivo sciatic nerve degeneration model and that this protective effect persists for at least 7 days following injury. These biological findings are consistent with the enzymatic inhibition of SARM1 observed in vitro and show that boldine suppresses axonal degeneration pathways.

Boldine Inhibits SARM1 NADase Activity in a Fluorescence Polarization Assay. To determine whether boldine directly inhibits SARM1 enzymatic activity, NAD⁺ hydrolysis was quantified using the Transcreener ADPR fluorescence polarization (FP) assay. This assay measures accumulation of ADPR produced during SARM1-mediated NAD⁺ cleavage.

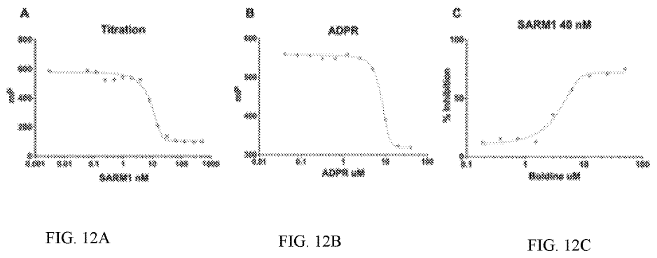

Enzyme titration experiments were first performed to identify assay conditions that maintained the reaction within the linear initial velocity range (<20% substrate conversion). Increasing SARM1 concentrations produced a robust and dose-dependent change in fluorescence polarization signal, with an EC50 of approximately 2.5 nM, confirming appropriate assay sensitivity and dynamic range (FIG. 12A).

To permit quantitative conversion of polarization signal to product formation, an ADPR standard curve was generated across a range of concentrations. The resulting calibration curve demonstrated a predictable reduction in polarization signal with increasing ADPR concentrations, validating the assay for accurate measurement of enzymatic activity (FIG. 12B).

Using optimized assay conditions, boldine was evaluated for its ability to inhibit SARM1 NADase activity. Dose-response analysis revealed concentration-dependent inhibition of enzyme activity, yielding an IC50 of approximately 7.5 μM when measured using 40 nM SARM1 (FIG. 12C). These results demonstrate that boldine directly suppresses SARM1 catalytic activity in vitro.

Structural Organization of SARM1 in Active and Inactive Conformations. To provide structural context for potential ligand binding interactions, previously solved cryo-electron microscopy structures of SARM1 representing inactive and activated conformational states were evaluated. Structural models were visualized and rendered using UCSF Chimera.

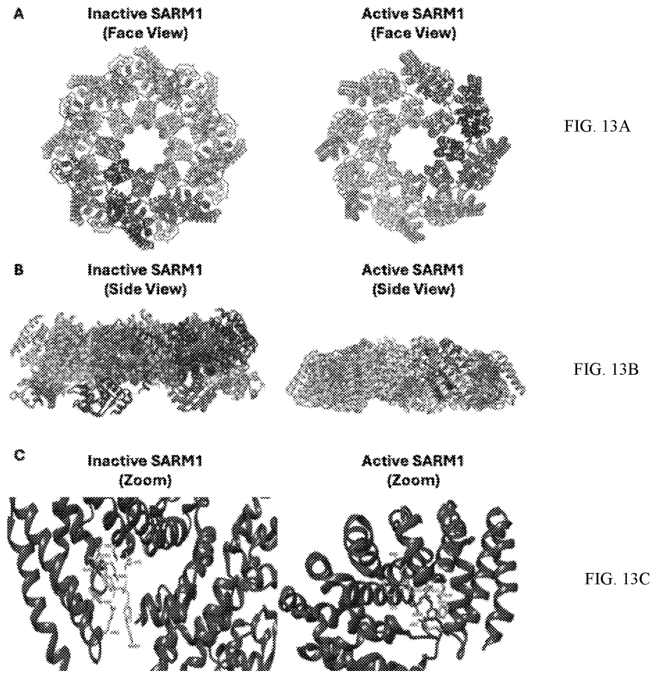

The autoinhibited form of SARM1 (PDB ID: 7CM6) forms an octameric ring complex in which the ARM regulatory domains create a peripheral ring surrounding the central catalytic domains. In this configuration, the TIR NADase domains are spatially restrained, preventing formation of the catalytically active interface (FIG. 13A).

Side-view representations highlight the stacked architecture of the ARM, SAM, and TIR domains within the octameric assembly (FIG. 13B). Examination of the regulatory ARM region reveals several surface-accessible pockets that may accommodate small molecule ligands capable of modulating conformational dynamics (FIG. 13C). Collectively, these structures provide a structural framework for evaluating potential ligand-binding sites.

Docking Simulations Identify Binding Sites Across Multiple SARM1 Conformations. To assess potential structural interactions between boldine and SARM1, molecular docking simulations were performed using several experimentally resolved SARM1 structures representing distinct conformational and domain states, including the autoinhibited full-length structure (PDB ID: 7CM6), the activated full-length structure (PDB ID: 7NAL), the isolated ARM regulatory domain (PDB ID: 7M6K), and the catalytic TIR NADase domain (PDB ID: 6O0Q).

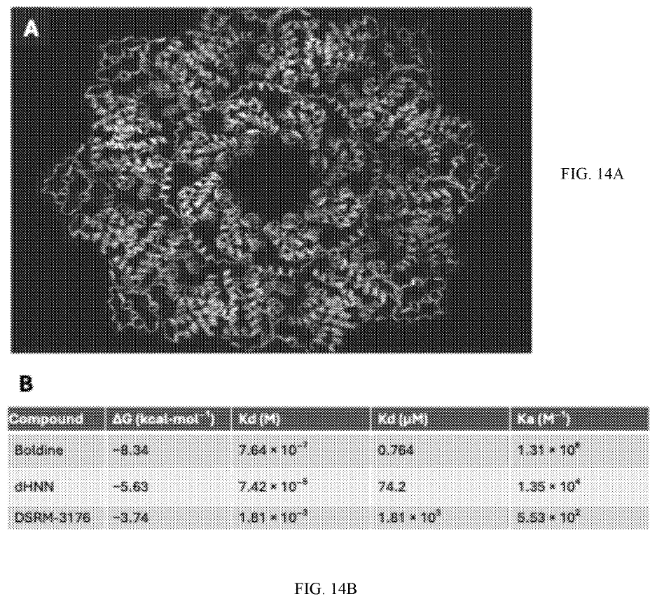

Docking simulations using the autoinhibited SARM1 structure (PDB ID: 7CM6) predicted a favorable binding pose for boldine within a pocket located near the ARM regulatory domain (FIG. 14A). Comparative docking analysis indicated that boldine exhibited the strongest predicted binding affinity among the compounds tested, including the reference inhibitor DSRM-3716 (FIG. 14B). The predicted interaction yielded a binding free energy of ΔG≈−8.3 kcal/mol, corresponding to an estimated Kd≈0.76 μM.

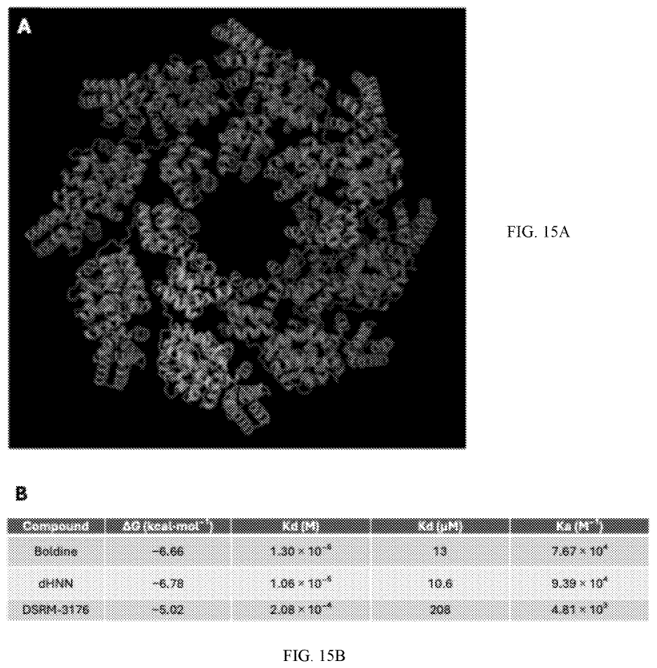

To determine whether boldine interactions were influenced by SARM1 conformational state, docking simulations were also performed using the activated full-length structure (PDB ID: 7NAL). In this conformation, displacement of the ARM domains permits oligomerization of the TIR catalytic domains into a configuration capable of NAD⁺ hydrolysis. Docking analysis identified ligand-binding pockets within the activated structure (FIG. 15A), although predicted binding affinities were reduced relative to those observed for the autoinhibited conformation. The highest-scoring pose for boldine yielded ΔG≈−6.6 kcal/mol, corresponding to an estimated Kd≈13 μM (FIG. 15B).

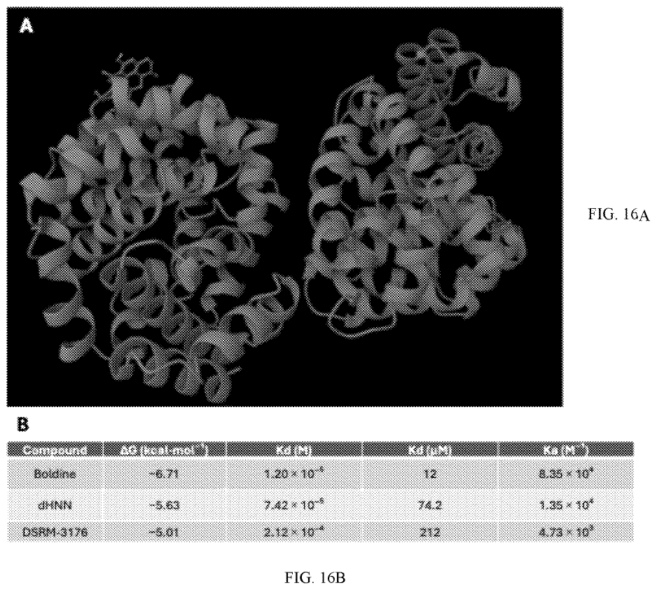

Additional docking simulations were performed using the isolated ARM regulatory domain (PDB ID: 7M6K). Boldine was predicted to occupy a surface-accessible pocket within the ARM domain consistent with potential regulatory ligand interactions (FIG. 16A). The highest-scoring docking configuration produced ΔG≈−6.7 kcal/mol, corresponding to an estimated Kd≈12 μM. Comparative docking analysis again identified stronger predicted binding affinity for boldine relative to DSRM-3716 under these conditions (FIG. 16B).

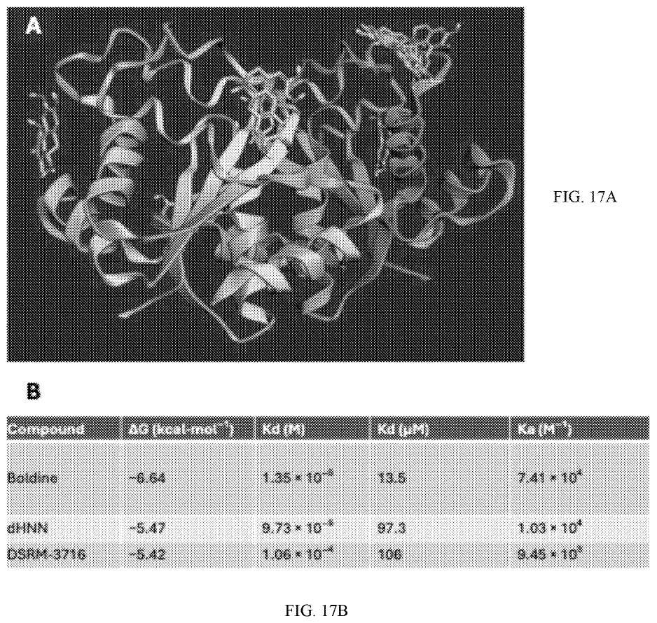

Finally, docking simulations using the isolated catalytic TIR domain (PDB ID: 6O0Q) identified several energetically favorable binding poses for boldine within pockets located near the catalytic region (FIG. 17A). The highest-scoring pose yielded ΔG≈−6.6 kcal/mol, corresponding to an estimated Kd≈13.5 μM. Comparative docking metrics shown in FIG. 17B indicate stronger predicted affinity for boldine relative to the reference compound.

The predicted ligand orientation places boldine adjacent to residues associated with catalytic activity, including Glu642, His685, and Asn640. However, the predicted binding pose does not directly occlude the NAD⁺ binding cleft, suggesting that inhibition may occur through indirect structural or conformational modulation rather than competitive substrate blockade.

Notably, the experimentally observed inhibition potency (IC50≈7.5 μM) is within one order of magnitude of the predicted binding affinities derived from docking simulations (Kd≈0.76-13 μM), supporting the plausibility of the predicted binding interactions.

Methods. Protein Structure Preparation. Protein structures for SARM1 were obtained from the Protein Data Bank (PDB) and included: the inactive octameric complex (PDB: 7CM6), activated full-length enzyme (PDB: 7NAL), isolated ARM regulatory domain (PDB: 7M6K), and TIR catalytic domain (PDB: 6O0Q) (C. Shen, M et al., Proc Natl Acad Sci USA 118, (2021); M. Sporny, et al. Elife 9, (2020); Y. Huang, et al., Proc Natl Acad Sci USA 122, e2424906122 (2025); and S. Horsefield, et al. Science 365, 793-799 (2019)). Additionally, structural ensembles were generated using the AlphaFold3-based Boltz-1 model accessed via the Neurosnap computational platform (P. H. Niu, et al. Zhonghua Yu Fang Yi Xue Za Zhi 59, 1156-1163 (2025)). Protein structures were prepared for docking by removing water molecules, adding hydrogen atoms, and assigning Gasteiger partial charges using UCSF Chimera (E. F. Pettersen, et al. J Comput Chem 25, 1605-1612 (2004)). Missing loops and side chains were modeled using MODELLER (B. Webb, A. Sali, Curr Protoc Bioinformatics 54, 5 6 1-5 6 37 (2016)).

Ligand Preparation. The three-dimensional structure of boldine was obtained from PubChem (CID: 10819) and energy-minimized using the MMFF94 force field in Avogadro. Rotatable bonds were identified and torsional degrees of freedom were assigned. Control compounds (dehydronitrosonisoldipine, DSRM-3716, carbenoxolone, calmidazolium) were prepared using the same protocol.

Molecular Docking Protocol. Molecular docking was performed using GNINA version 1.3, a deep learning framework that employs 3D convolutional neural networks (CNNs) for scoring protein-ligand interactions. An exhaustiveness parameter of 8 was employed, and 10 binding poses per ligand were generated. For blind docking experiments on the TIR domain, the entire domain surface was included in the search space. For targeted docking to specific pockets, the search box was centered on important residues identified from structural analysis (e.g., Glu642, His685, Asn640 for the NAD+ binding region).

Scoring and Thermodynamic Calculations. Binding affinities were predicted using the CNN scoring function implemented in GNINA27. Thermodynamic parameters were calculated using the equations: ΔG=−RT ln(Ka); Kd=1/Ka where R is the gas constant (1.987 cal·mol⁻¹·K⁻¹), T is the temperature (298 K). T is the temperature (298 K), and Ka is the association constant. The best-scored pose for each ligand was selected for detailed structural analysis and visualization in UCSF Chimera and PyMOL (E. F. Pettersen, et a., J Comput Chem 25, 1605-1612 (2004)).

Validation of Docking Protocol. The docking protocol was validated by re-docking known SARM1 inhibitors (DSRM-3716) and comparing predicted Kd values with experimentally determined binding constants reported in the literature (R. O. Hughes, et al., Cell Rep 34, 108588 (2021)). The root-mean-square deviation (RMSD) between predicted and crystallographic poses was <2.0 Å, confirming the reliability of the method.

Assay principle. SARM1 NAD hydrolase activity was quantified using a far-red competitive fluorescence polarization (FP) immunoassay (Transcreener ADPR FP Assay). In this format, SARM1 cleaves NAD to generate ADPR; ADPR is converted in real time to AMP by an ADPR-AMP coupling enzyme, and the resulting AMP competes with an AMP2/GMP2 fluorescent tracer for binding to an AMP2/GMP2 antibody, producing a measurable FP signal change. The assay is intended for biochemical measurements using purified enzyme.

Reagents and instrumentation. Recombinant human SARM1 (BPS Bioscience, Cat #100069) was used with assay buffer consisting of 50 mM Tris-HCl (pH 7.5), 5 mM MgCl₂, and 0.01% Triton X-100, prepared in nuclease-free water. Reactions were performed in black 384-well plates and read on an FP-capable plate reader (iD5 Molecular Devices).

Reaction setup. Enzyme reactions were assembled at 10 μL total volume (5 μL enzyme/buffer+5 μL substrate/coupling mix) and then converted to a 20 μL “complete assay” by adding 10 μL of 1×AMP Detection Mix. NAD was used at 20 μM final (from a 40 μM working concentration in the substrate/coupling mix) with coupling enzyme at 1× in the enzyme reaction. The detection mix yielded final “complete assay” concentrations of 0.5× Stop & Detect Buffer B, 4 nM AMP2/GMP2 tracer, and 7.5 μg/mL AMP2/GMP2 antibody. Plates were mixed (~40 s), sealed, incubated 60 min at 30° C. for the enzyme reaction, then incubated 90 min at room temperature after addition of detection mix prior to FP measurement.

Enzyme titration, ADPR standard curve, and boldine (inhibitor) dose response. To identify an appropriate enzyme concentration, SARM1 was titrated by two-fold serial dilution across wells, aiming to remain in the initial velocity regime (<20% substrate conversion) and selecting a concentration that produced ~80% of maximal signal. For quantitative conversion of FP signal to product formed, a 11-point ADPR standard curve was generated by serially diluting ADPR (starting from 40 μM working) to cover a range corresponding to 20 μM down to 0 μM. For inhibitor profiling, SARM1 was preincubated with test compounds (30 min, room temperature) before initiating reactions with substrate/coupling mix and proceeding as above; IC50 values were obtained from dose-response curves.

Ex Vivo Nerve Culture and Axon Degeneration Assay. Adult male Sprague-Dawley rats (300-330 g, Charles River Laboratories) were used for the ex vivo and in vivo experiments.

Sciatic Nerve Harvest and Culture. Rats were euthanized by CO2 asphyxiation followed by decapitation. Sciatic nerves were rapidly dissected from the mid-thigh to the trifurcation under sterile conditions and placed in ice-cold Hanks' Balanced Salt Solution (HBSS). Nerves were transected into 10 mm segments and transferred to 24-well culture plates containing DMEM supplemented with 10% fetal bovine serum (FBS), 100 U/mL penicillin, and 100 mg/mL streptomycin. Cultures were maintained at 37° C. in a humidified atmosphere of 5% CO2.

Boldine Treatment. Boldine was obtained from two independent sources: Sigma-Aldrich (catalog #B3916, 98% purity by HPLC) and Kimun Pharma. Stock solutions (100 mM) were prepared in dimethyl sulfoxide (DMSO) and diluted to working concentrations (10-100 mM) in culture medium (final DMSO concentration <0.1%). Vehicle control cultures received an equivalent volume of DMSO. Media were changed daily with fresh boldine or vehicle for the duration of the experiment (3 or 7 days).

Immunofluorescence Staining and Quantification. At the indicated time points, nerve explants were fixed in 4% paraformaldehyde (PFA) in phosphate-buffered saline (PBS) for 1 hour at room temperature, cryoprotected in 30% sucrose overnight at 4° C., and embedded in OCT compound. Cryosections (20 μm) were cut on a cryostat, mounted on Superfrost Plus slides, and stored at −80° C. until use. For immunostaining, sections were permeabilized with 0.3% Triton X-100 in PBS for 10 minutes, blocked with 5% normal horse serum (NHS) for 1 hour, and incubated overnight at 4° C. with chicken neurofilament-light conjugated to Alexa-Fluorophore (NF-L, 1:500). Sections were mounted with Fluoromount-G and imaged on a confocal microscope.

Axon Density Quantification. Neurofilament-positive area was quantified using ImageJ by thresholding fluorescence intensity and calculating the percentage of positive area relative to total nerve cross-sectional area (three sections per nerve, n=3 nerves per group) (C. A. Schneider, et al. Nat Methods 9, 671-675 (2012)). Data are presented as mean±SEM. Statistical comparisons were performed using one-way ANOVA with Tukey's post-hoc test; p<0.05 was considered statistically significant.

1. A method of inhibiting sterile alpha and TIR motif-containing protein 1 (SARM1) NADase activity in an axon, the method comprising: contacting the axon with an effective amount of a SARM1 inhibitor, thereby inhibiting SARM1 NADase activity in the axon.

2. The method of claim 1, wherein the contacting step occurs for a period of at least 1 hour, at least 6 hours, at least 24 hours, at least 72 hours, or continuously for four or more days.

3. The method of claim 1, wherein the SARM1 inhibitor is selected from the group consisting of a isoquinoline alkaloid, an isothiazole, a benzisothiazole, and an isothiazolinone derivative.

4. The method of claim 3, wherein the isoquinoline alkaloid is boldine.

5. The method of claim 1, wherein the SARM1 inhibitor is dehydronitrosonisodipine (dHNN) or DSRM-3716.

6. The method of claim 4, wherein boldine inhibits SARM1 NADase activity by binding to a catalytic domain of SARM1 or regulatory domain of SARM1.

7. The method of claim 1, wherein the contacting step is performed ex vivo, in vitro, in situ, or in vivo.

8. The method of claim 1, wherein the contacting step is by local delivery or regionally-restricted delivery of the SARM1 inhibitor to the axon.

9. (canceled)

10. The method of claim 1, wherein the SARM1 inhibitor is delivered locally to the axon at a site of injury.

11.-20. (canceled)

21. A method of treating or preventing degeneration of an injured axon, the method comprising: contacting a neural tissue comprising the injured axon ex vivo with an effective amount of a sterile alpha and TIR motif-containing protein 1 (SARM1) inhibitor, thereby treating or preventing degeneration in the injured axon.

22.-24. (canceled)

25. The method of claim 21, wherein the neural tissue is maintained ex vivo in the presence of the SARM1 inhibitor for a period of 1 minute to 14 days prior to transplantation.

26.-29. (canceled)

30. The method of claim 25, wherein the SARM1 inhibitor is in a biomaterial matrix.

31. The method of claim 30, wherein the biomaterial matrix is a hydrogel, a polymer scaffold, a fibrin matrix, a collagen matrix, or a synthetic extracellular matrix material.

32. (canceled)

33. The method of claim 21, wherein the SARM1 inhibitor is selected from the group consisting of a isoquinoline alkaloid, an isothiazole, a benzisothiazole, and an isothiazolinone derivative.

34. The method of claim 33, wherein the isoquinoline alkaloid is boldine.

35. The method of claim 21, wherein the SARM1 inhibitor is dehydronitrosonisodipine (dHNN) or DSRM-3716.

36. A method of enhancing fusion of one or more severed axons, the method comprising:
(a) contacting ex vivo a neural tissue comprising the one or more severed axons with an effective amount of a sterile alpha and TIR motif-containing protein 1 (SARM1) inhibitor; and
(b) contacting ex vivo the neural tissue comprising the two or more severed axons in (a) with an effective amount of a fusogen,
wherein the contacting in (a) is prior to or concurrent with the contacting of (b), thereby enhancing the fusion of the one or more severed axons.

37. (canceled)

38. The method of claim 36, wherein the SARM1 inhibitor is selected from the group consisting of a isoquinoline alkaloid, an isothiazole, a benzisothiazole, and an isothiazolinone derivative.

39. The method of claim 38, wherein the isoquinoline alkaloid is boldine.

40.-43. (canceled)

44. The method of claim 36, wherein the fusogen is a polyethylene glycol (PEG), chitosan, a fusogenic lipid, a membrane fusion peptide, or a viral fusogen.

45.-76. (canceled)
